(19) United States
(12) Patent Application Publication
(10) Pub. No.: US 2008/0175872 A1
(43) Pub. Date: Jul. 24, 2008

(54) Controlled Release Dosage Form Containing Lercanidipine and a Performance-enhancing Acid

(75) Inventors: Esteban A. Abalo, Buenos Aires (AR); Marcelo A. Ricci, Buenos Aires (AR); Glenn A. Meyer, Wilmington, NC (US); Pablo F. A. Carraud, Buenos Aires (AR)

Correspondence Address: INNOVAR, LLC, P O BOX 250647, PLANO, TX, 75025, US

(73) Assignee: OSMOTICA CORP., Tortola (VG)

(21) Appl. No.: 11/859,063

(22) Filed: Sep. 21, 2007

Related U.S. Application Data
(60) Provisional application No. 60/827,360, filed on Sep. 28, 2006.

(51) Int. Cl. A61K 9/00; A61K 31/44; A61P 9/00

(52) U.S. Cl. 424/400; 514/356

(57) ABSTRACT

A controlled release dosage form containing lercanidipine, or a salt thereof, a performance-enhancing acid, and at least one other pharmaceutical excipient exhibits enhanced in vitro dissolution of lercanidipine, enhanced storage stability based upon the reduced degradation of lercanidipine, and/or enhanced in vivo bioavailability of lercanidipine as compared to an otherwise similar controlled release dosage form excluding the performance-enhancing acid but containing the same amount of lercanidipine.

### CROSS-REFERENCE TO RELATED APPLICATIONS

This application is a continuation-in-part of and claims the priority of U.S. Provisional Application No. 60/827,360 filed Sep. 28, 2006, the entire disclosure of which is hereby incorporated by reference.

### FIELD OF THE INVENTION

This invention pertains to a controlled release dosage form for the controlled delivery of lercanidipine. More particularly, it pertains to an osmotic device that comprises lercanidipine, at least one swellable polymer, and a carboxylic acid. The invention also provides a method of treating a disorder or disease that is therapeutically responsive to lercanidipine.

### BACKGROUND OF THE INVENTION

Lercanidipine (methyl 1,1,N-trimethyl-N-(3,3-diphenylpropyl)-2-aminoethyl 1,4-dihydro-2,6-dimethyl-4-(3-nitrophenyl)pyridine-3,5-dicarboxylate) is a highly lipophilic dihydropyridine calcium antagonist (calcium channel blocker) with a long duration of action and high vascular selectivity. It has a high affinity for and competitively antagonizes the dihydropyridine subunit of the L-type calcium channel. Lercanidipine is primarily a vasodilator that lowers blood pressure by decreasing peripheral vascular resistance at the level of the small arterioles.

Lercanidipine is subject to photochemical degradation when exposed to UV-A radiation, and it is also subject to degradation and oxidation in solution.

U.S. Pat. No. 4,705,797 to Nardi et al., U.S. Pat. No. 5,767,136 to Sartani et al., U.S. Pat. No. 4,968,832 to Bianchi et al., U.S. Pat. No. 5,912,351 to Leonardi et al., and U.S. Pat. No. 5,696,139 to Leonardi et al., describe lercanidipine along with methods for its preparation and its resolution into the individual enantiomers.

The hydrochloride salt of lercanidipine has been approved for the treatment of hypertension and has been marketed since 1996 in several European countries under the trademark Zanidip™ (Recordati S.p.A. (Milan, Italy)). The recommended starting oral dose of lercanidipine HCl is 10 mg once daily and is increased after at least 2 weeks, if necessary, to 20 mg daily. Upon oral administration of an immediate release form of lercanidipine, peak plasma level (Tmax) occurs 1-3 hours following administration.

U.S. Pat. No. 6,852,737 to Bonifacio et al. discloses that lercanidipine hydrochloride shows polymorphic features and crystallizes into different crystalline forms depending on the process followed and on the solvents used. Crude lercanidipine hydrochloride Form (A), which has a melting point of about 150-152° C. (DSC peak) and comprises about 3-4% (w/w) ethyl acetate, crude lercanidipine hydrochloride Form (B) which has a melting point of about 131-135° C. (DSC peak) and comprises about 0.3-0.7% (w/w) ethyl acetate, lercanidipine hydrochloride crystalline Form (I), and lercanidipine hydrochloride crystalline Form (II) are provided.

U.S. Pregrant Pub. No. 2003/0069285 to Leonardi et al. discloses solvates of lercanidipine hydrochloride with organic solvents and lercanidipine hydrochloride crystalline forms obtained from said solvates, such as lercanidipine hydrochloride crystalline Form (III) and (IV).

U.S. Pregrant Pubs. No. 2003/0083355, No. 2004/0204459, and No. 2005/0239847 all to Bonifacio et al. disclose novel lercanidipine crude Forms (A), and (B) and novel lercanidipine hydrochloride crystalline Forms (I) and (II) obtained from said crude Forms.

U.S. Pregrant Pub. No. 2006/0047125 to Leonardi et al. discloses new addition salts of lercanidipine comprising lercanidipine and an acid counterion. The acid counterion is selected from the group consisting of: (i) inorganic acids, (ii) sulfonic acids, (iii) monocarboxylic acids, (iv) dicarboxylic acids, (v) tricarboxylic acids, and (vi) aromatic sulfonimides, with the proviso that said acid counterion is not hydrochloric acid.

U.S. Pregrant Pub. No. 2006/0073200 to Leonardi et al. discloses a modified release lercanidipine pharmaceutical composition comprising at least one waxy substance and a therapeutically effective amount of lercanidipine. The oral administration of the modified release lercanidipine pharmaceutical composition contained in capsules to a patient results in a mean lercanidipine plasma concentration of greater than 0.5 ng/ml for the full time period of about 24 hours after administration of the composition to the patient.

U.S. Pregrant Pub. No. 2006/0165788 to Abramowitz et al. discloses a modified release composition that release pulses of lercanidipine based on the pH of the use environment.

U.S. Pregrant Pub. No. 2006/0165789 to Abramowitz et al. discloses a modified release bead composition which provides modified release of lercanidipine independent of pH and therefore provides release of lercanidipine even upon exposure to the low pH use environments, such as gastric fluid.

PCT International Application Publication No. WO 05/053689 discloses a pharmaceutical composition comprising lercanidipine or an analog or a pharmaceutically acceptable salt thereof as an active substance and a pharmaceutically acceptable vehicle. The composition upon oral administration to a mammal in need thereof releases the active substance in a controlled manner.

Lercanidipine and its salts are virtually insoluble in water, with an aqueous solubility of about 5 g/ml. Lercanidipine is essentially insoluble in gastrointestinal pH range of 1 to 8. Lercanidipine is classified as a low permeable drug, as defined by the FDA, and displays extensive presystemic first pass elimination, as a result of its being a substrate for cytochrome P450 IIIA4 isoenzyme. Lercanidipine administered in the absence of food is not entirely absorbed which results in low and variable bioavailability. The dependence of effective dosing and absorption of lercanidipine upon co-administration of food is undesirable due to fluctuations in effectiveness, inter-patient variability, and poor patient acceptance and compliance.

Given the importance of lercanidipine in the treatment of various disorders, there remains a need in the art for improved lercanidipine oral dosage forms.

### SUMMARY OF THE INVENTION

The controlled release solid dosage form of the invention seeks to overcome one or more disadvantages present in other dosage forms containing lercanidipine. The controlled release solid dosage form of the present invention overcomes the difficulties caused by the low solubility of lercanidipine in aqueous media thereby providing good lercanidipine absorption and bioavailability over a period of at least 24 hours compared to currently available lercanidipine compositions.

In one aspect, the dosage form is an osmotic device comprising: 1) a core comprising lercanidipine, or a pharmaceutically acceptable salt thereof, in admixture with a performance-enhancing acid and one or more other pharmaceutical excipients; and 2) a wall enveloping the core and comprising at least one preformed passageway. The dosage form provides a controlled release of lercanidipine over a period of about 8-36 hours, about 10-30 hours, or about 12-24 hours, or about 18-24 hours.

Some embodiments of the invention comprise a salt of lercanidipine. The salt can be selected from mineral acid or organic acid salts of lercanidipine. In some embodiments, the performance-enhancing acid is an organic acid and the acid used to form the salt of the lercanidipine is a mineral acid. In some embodiments, the organic acid is a non-aromatic carboxylic acid; a monocarboxylic acid, such as acetic acid, (+)-L-lactic acid, DL-lactic acid, DL-mandelic acid, gluconic acid, cinnamic acid, salicylic acid, and gentisic acid; a dicarboxylic acid, such as oxalic acid, 2-oxo-glutaric acid, malonic acid, (−)-L-malic acid, mucic acid, (+)-L-tartaric acid, fumaric acid, succinic acid, maleic acid, and terephthalic acid; a hydroxy-carboxylic acid; a hydroxy-dicarboxylic acid; a tricarboxylic acid, such as citric acid, or aromatic carboxylic acid; sulfonic acids, such as methanesulfonic acid, benzenesulfonic acid, toluenesulfonic acid, and napthalene-1,5-disulfonic acid; alpha-hydroxy acids such as tartaric acid, citric acid, ascorbic acid and malic acid; and aromatic sulfonimides such as saccharin. In some embodiments, the mineral acid is hydrochloric acid, hydrobromic acid, sulfuric acid, sulfonic acid, sulfamic acid, phosphoric acid and nitric acid. Specific pharmaceutically acceptable salts of lercanidipine, include but are not limited to, the hydrochloride, besylate and napadisylate salts. The performance-enhancing acid can be, as described herein, a monocarboxylic acid, a dicarboxylic acid, a tricarboxylic acid, a hydroxy-carboxylic acid, a hydroxy-dicarboxylic acid, a hydroxy-tricarboxylic acid, an alpha-hydroxycarboxylic acid or a non-aromatic organic acid.

Another aspect of the invention provides an osmotic device, wherein the core thereof comprises lercanidipine (or a pharmaceutically acceptable salt thereof), a performance-enhancing acid, an osmotic agent, and at least one swelling polymer, and optionally one or more other materials, e.g. pharmaceutical excipients, as discussed herein.

The dosage form of the invention can be used to treat a disease or disorder that is therapeutically responsive to lercanidipine. By therapeutically responsive disease or disorder is meant that a subject suffering from such a disease or disorder will enjoy a clinical benefit upon administration of one or more osmotic devices of the invention according to a defined dosing regimen. Exemplary therapeutically responsive diseases or disorder include hypertension, fibrinolysis, atherosclerosis, coronary heart disease (e.g., chronic stable angina, myocardial infarction), congestive heart failure, and cerebrovascular diseases such as cerebral infarction and cerebral apoplexy. The osmotic device of the invention can also be used to improve memory in a subject and reduce the incidence of stroke. The dosage form can contain a therapeutically effective amount of lercanidipine such that a single or two or more dosage forms will together result in a unit dose of lercanidipine. Any suitable dosing regimen can be used. The osmotic device can be administered once, twice, or three times daily, weekly, biweekly, monthly, bimonthly, quarterly, semiannually, annually, or a combination thereof as required to provide the desired clinical benefit to a subject.

The dosage form can further comprise at least one other pharmaceutically active agent (drug), whereby the dosage form can comprise two or more different drugs. Such a dosage form would be useful for the treatment of a disease or disorder that is therapeutically responsive to lercanidipine and/or any other drug(s) present in the dosage form.

Other features, advantages and embodiments of the invention will become apparent to those skilled in the art by the following description, and accompanying examples.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts a lercanidipine in vitro dissolution profile for the osmotic device described in Example 1.

FIG. 2 depicts the predicted release profiles of the osmotic device containing lercanidipine 30 mg strength in the core and lercanidipine 10 mg strength in an immediate or rapid release external drug-containing coat disclosed in Example 5.

FIG. 3 depicts the predicted release profiles of the osmotic device containing lercanidipine 50 mg strength in the core and lercanidipine 10 mg strength in an immediate or rapid release external drug-containing coat disclosed in Example 5.

FIG. 4 depicts the release profile of the osmotic device containing fumaric acid disclosed in Example 6.

FIG. 5 depicts the release profile of the osmotic device containing oxalic acid disclosed in Example 6.

FIG. 6 depicts the release profile of the osmotic device containing succinic acid disclosed in Example 6.

FIG. 7 depicts the release profile of the osmotic device containing tartaric acid disclosed in Example 6.

FIG. 8 depicts the release profile of the osmotic device containing citric acid disclosed in Example 6.

FIG. 9 depicts the release profile of the osmotic device containing lercanidipine 30 mg strength in the core and lercanidipine 10 mg strength in an immediate or rapid release external drug-containing coat disclosed in Example 8.

FIG. 10 depicts the release profile of the osmotic device containing lercanidipine 50 mg strength in the core and lercanidipine 10 mg strength in an immediate or rapid release external drug-containing coat disclosed in Example 8.

### DETAILED DESCRIPTION OF THE INVENTION

The invention may be better understood by reference to the following definitions provided herein.

By “performance-enhancing acid” is meant an organic acid, in the controlled release dosage form, that enhances the bioavailability of lercanidipine when administered to a subject in need thereof and/or that exhibits reduced degradation (increased stability) of lercanidipine during storage, as compared to a control osmotic device excluding the performance-enhancing acid. A performance-enhancing acid may also increase the rate of release of lercanidipine or increase the overall amount of lercanidipine released upon exposure to an aqueous environment of use or following administration to a subject in need thereof as compared to an otherwise similar (control) osmotic device excluding the performance-enhancing acid. As a result, the performance-enhancing acid enhances the in vitro and/or in vivo performance of the controlled release dosage form, whereby the dosage form provides an improved bioavailability of lercanidipine or possesses an improved storage stability (improved shelf-life based upon the reduced degradation of lercanidipine) as compared to an otherwise similar dosage form containing the same amount of lercanidipine but excluding the performance-enhancing acid.

A performance-enhancing acid can comprise a monocarboxylic acid, dicarboxylic acid, tricarboxylic acid, hydroxy-carboxylic acid, hydroxy-dicarboxylic acid, hydroxy-tricarboxylic acid, nonaromatic organic acid, or a combination thereof. In some embodiments, the performance-enhancing acid is citric acid, maleic acid, ascorbic acid, fumaric acid, oxalic acid, succinic acid or a combination thereof.

By “immediate release” is meant a release of an active agent to an environment over a period of seconds to no more than about 30 minutes once release has begun and release begins within a second to no more than about 15 minutes after administration.

By “rapid release” is meant a release of an active agent to an environment over a period of 1-59 minutes or 1 minute to three hours once release has begun and release can begin within a few minutes after administration or after expiration of a delay period (lag time) after administration.

By “controlled release” is meant a release of an active agent to an environment over a period of about eight hours up to about 12 hours, 16 hours, 18 hours, 20 hours, a day, or more than a day. A controlled release can begin within a few minutes after administration or after expiration of a delay period (lag time) after administration.

By “sustained release” is meant a controlled release of an active agent to maintain a constant drug level in the blood or target tissue of a subject to which the device is administered.

By “extended release” is meant a controlled release of an active agent from a dosage form to an environment over an extended period of time. As used herein, the term “extended release” profile assumes the definition as widely recognized in the art of pharmaceutical sciences. An extended release dosage form will release drug at substantially constant rate over an extended period of time or a substantially constant amount of drug will be released incrementally over an extended period of time. The term “extended release”, as regards to drug release, includes the terms “controlled release”, “prolonged release”, “sustained release”, or “slow release”, as these terms are used in the pharmaceutical sciences.

A delayed but controlled release dosage form is one that provides a delayed release of a drug followed by a controlled release of the drug. By delayed release is meant any formulation technique wherein release of the active substance from the dosage form is modified to occur at a later time than that from a conventional immediate release product. In other words, the beginning of the controlled release of drug is delayed by an initial period of time. The period of delay is generally about 5 minutes to 10 hours, or 30 minutes to 10 hours, or 1 hour to 10 hours.

A zero-order release profile characterizes the release profile of a dosage form that releases a constant amount of drug per unit time. A pseudo-zero order release profile is one that approximates a zero-order release profile. A dissolution curve shows a zero or pseudo-zero order release profile if its release rate remains constant (or relatively constant within ±10% of the average value) in the interval of time 0≦a<t≦b. Any profile following the equation:

(M(t)/Mᵣ)=k(t−a)ⁿ 0.9≦n≦1.1

has the following release rate equation:

(1/M)(dM/dt)=kn(t−a)ⁿ⁻¹

A sigmoidal release profile characterizes the release profile of a dosage form that releases a drug in a controlled manner but very slowly during a first release period, then more rapidly during a second release period and finally very slowly during a third release period such that the release profile resembles a sigmoid. A dissolution curve shows a sigmoid release profile within a certain interval of time 0≦a<t≦b if its release rate reaches a single maximum within the interval (a, b) excluding the extremes. That is equivalent to consider a point of time T* so that the release rate is an increasing function of time for a≦t<T* and a decreasing function of time, as determined by the following equation:

### Weibull Function

(M(t)/M_{T})=W_{inf{}1−exp{−[(t−tᵢ)/β]^{α}}} Parameter ranges:

t₁: between 0 and 3
β: between 7 and 12
α: 1<α<3
W_{inf}: between 0.5 and 1.1

A first order release profile characterizes the release profile of a dosage form that releases a percentage of a drug charge per unit time. A pseudo-first order release profile is one that approximates a first order release profile. A dissolution curve shows a first or pseudo-first order release profile within a certain interval of time 0≦a<t≦b if its release rate is a continue monotone decreasing function of time. Specifically, a dissolution curve shows a first order profile whenever its release rate is proportional to the remaining undissolved amount of drug, as determined by the following equation:

(M(t)/M_{T})=1−exp(−kt)

A dissolution curve shows a pseudo-first order profile when the drug release rate decreases with time as described by the Fickian or anomalous Fickian diffusion controlled release equation:

(M(t)/M_{T})=ktⁿ, 0.3≦n≦0.7

By “unitary core” is meant the core of an osmotic device that is not divided into two or more layers or laminas. The core is considered to be the composition enclosed within the wall, e.g. semipermeable membrane, of the osmotic device. The ingredients of the core may be present as a heterogeneous mixture or homogeneous mixture. A homogeneous mixture is one wherein all of the ingredients have been thoroughly mixed such that the composition of the formulation is substantially the same throughout different portions of the core. The combined step of mixing and directly compressing the ingredients of the core generally provides a homogeneous mixture. A heterogeneous mixture is one wherein the ingredients of the core are divided into two or more groups that are processed separately to form two or more respective blends, at least one of which contains drug and at least one of which contains the osmotic agent. The blends are then mixed together and compressed to form the unitary core. A heterogeneous mixture can be obtained by wet granulation, dry granulation, pelleting or combinations thereof.

The core of the osmotic device of the present invention comprises lercanidipine (or a pharmaceutically acceptable salt thereof), a performance-enhancing acid (for example, citric acid), an osmotic agent, and at least one swelling polymer, and can further comprise one or more other materials, e.g. pharmaceutical excipients, as discussed herein.

The osmotic device of the invention can comprise osmotically effective solutes or osmotic agents, i.e. osmagents, that are capable of being totally or partially solubilized in the fluid. These osmagents can aid in either the suspension or dissolution of lercanidipine from the core. Exemplary osmagents include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, magnesium succinate, sodium succinate, sodium butyrate, sodium fumarate, sodium benzenesulfonate, sodium toluenesulfonate, sodium methanesulfonate, combinations thereof and other similar or equivalent materials which are widely known in the art. U.S. Pat. No. 4,077,407 to Theeuwes et al., the entire disclosure of which is hereby incorporated by reference, discloses suitable osmagents.

One or more osmopolymers can also be included in the core of the device to aid in the delivery of lercanidipine. Osmopolymers are well known to those of ordinary skill in the osmotic device art and well described in the patent and scientific literature. Exemplary osmopolymers include hydrophilic polymers that swell upon contact with water. Osmopolymers may be of plant or animal origin, or synthetic. Examples of osmopolymers include: poly(hydroxy-alkyl methacrylates) with molecular weight of 30,000 to 5,000,000, poly(vinylpyrrolidone) with molecular weight of 10,000 to 360,000, anionic and cationic hydrogels, polyelectrolyte complexes, poly(vinyl alcohol) having low acetate residual, optionally cross-linked with glyoxal, formaldehyde or glutaraldehyde and having a degree of polymerization of 200 to 30,000, a mixture of methyl cellulose, cross-linked agar and carboxymethylcellulose, a mixture of hydroxypropyl methylcellulose and sodium carboxymethylcellulose, sodium carboxymethylcellulose, hydroxypropyl methylcellulose, polyethylene oxide, polymers of N-vinyllactams, polyoxyethylene-polyoxypropylene gels, polyoxybutylene-polyethylene block copolymer gels, carob gum, polyacrylic gels, polyester gels, polyurea gels, polyether gels, polyamide gels, polypeptide gels, polyamino acid gels, polycellulosic gels, carbopol acidic carboxy polymers having molecular weights of 250,000 to 4,000,000, Cyanamer polyacrylamides, cross-linked indene-maleic anhydride polymers, Good-Rite™ polyacrylic acids having molecular weights of 80,000 to 200,000, Polyox™ polyethylene oxide polymers having molecular weights of 100,000 to 5,000,000, starch graft copolymers, and Aqua-Keeps™ acrylate polymer polysaccharides. These materials swell or expand to an equilibrium state when exposed to water or other biological fluids. This volume expansion is used to physically force the pharmaceutical agent out through openings that have been formed in the wall, shell or coating during manufacture. Exemplary osmopolymers are disclosed in U.S. Pat. No. 5,422,123; U.S. Pat. No. 4,783,337; U.S. Pat. No. 4,765,989; U.S. Pat. No. 4,612,008; U.S. Pat. No. 4,327,725; U.S. Pat. No. 4,609,374; U.S. Pat. No. 4,036,228; U.S. Pat. No. 4,992,278; U.S. Pat. No. 4,160,020; U.S. Pat. No. 4,615,698. The osmopolymers generally swell or expand to a very high degree, usually exhibiting a 2 to 60 fold volume increase. The osmopolymers can be non-cross-linked or cross-linked. The swellable, hydrophilic polymers are, in one embodiment, lightly cross-linked, such as cross-links being formed by covalent or ionic bonds.

The semipermeable membrane of the osmotic device is formed of a material that is substantially permeable to the passage of fluid from the environment of use to the core and substantially impermeable to the passage of active agent from the core. Many common materials that form a semipermeable wall which are known by those of ordinary skill in the art of pharmaceutical sciences are suitable for this purpose. Exemplary materials are cellulose esters, cellulose ethers and cellulose esters-ethers. However, it has been found that a semipermeable membrane comprising cellulose acetate (CA) and poly(ethylene glycol) (PEG), in particular PEG 400, performs well when used in combination with the other materials required in the present osmotic device. This particular combination of CA and PEG provides a semipermeable membrane that gives the osmotic device a well controlled release profile for the active agent in the core and that retains its chemical and physical integrity in the environment of use. The ratio of CA:PEG generally ranges from about 50-99% by weight of CA:about 50-1% by weight of PEG, and about 95% by weight of CA:about 5% by weight of PEG. The ratio can be varied to alter permeability and ultimately the release profile of the osmotic device. Other suitable materials can include a selected member of the group of cellulose acylates such as cellulose acetate, cellulose diacetate, cellulose triacetate and combinations thereof. Many suitable polymers, include those disclosed in Argentine Patent No. 199,301, U.S. Pat. No. 6,004,582 and references cited herein, the disclosures of which are hereby incorporated by reference.

Representative materials for making the semipermeable membrane include a member selected from the group consisting of cellulose acylate, cellulose diacylate, cellulose triacylate, cellulose acetate, cellulose diacetate, cellulose triacetate, mono, di and tricellulose alkanylates, mono, di and tricellulose aroylates, and the like. Exemplary polymers include cellulose acetate having a substitution degree (D.S.) up to 1 and an acetyl content up to 21%; cellulose acetate having an acetyl content of 32 to 39.8%; cellulose diacetate having a D.S. of 1 to 2 and an acetyl content of 21 to 35%; cellulose triacetate having a D.S. of 2 to 3 and an acetyl content of 35 to 44.8%; and the like. More specific cellulosic polymers include cellulose propionate having a D.S. of 1.8 and a propionyl content of 39.2 to 45% and a hydroxyl content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%; a butyryl content of 17 to 53% and a hydroxyl content of 0.5 to 4.7%; cellulose triacylates having a D.S. of 2.9 to 3 such as cellulose trivalerate, cellulose trilaurate, cellulose tripalmitate, cellulose trisuccinate, and cellulose trioclanoate; cellulose diacylates having a D.S. of 2.2 to 2.6 such as cellulose disuccinate, cellulose dipalmitate, cellulose dioclanoate, cellulose dipentale, and the like. Additional semipermeable polymers include acetaldehyde dimethyl acetate, cellulose acetate ethyl carbamate, cellulose acetate phthalate for use in environments having a low ph, cellulose acetate methyl carbamate, cellulose acetate dimethyl aminoacetate, semipermeable polyamides, semipermeable polyurethanes, semipermeable sulfonated polystyrenes, cross-linked selectively semipermeable polymers formed by the coprecipitation of a polyanion and a polycation as disclosed in U.S. Pat. No. 3,173,876, U.S. Pat. No. 3,276,586, U.S. Pat. No. 3,541,005, U.S. Pat. No. 3,541,006, and U.S. Pat. No. 3,546,142; semipermeable polymers as disclosed by Loeb and Sourirajan in U.S. Pat. No. 3,133,132; lightly cross-linked polystyrene derivatives; cross-linked poly(sodium styrene sulfonate), cross-linked poly(vinylbenzyltrimethyl ammonium chloride). These and others polymers are disclosed in U.S. Pat. No. 3,845,770, U.S. Pat. No. 3,916,899, U.S. Pat. No. 4,765,989 and U.S. Pat. No. 4,160,020; and in Handbook of Common Polymers (Scott, J. R. and Roff, W. J., eds.; 1971; CRC Press, Cleveland, Ohio).

The cellulose esters differ in their cellulose chain length and the type and amount of ester groups attached to the chain. For cellulose acetates, as the amount of acetyl content increases, the permeability decreases. The cellulose acetate grade 1 comprises 7-10% by weight of hydroxyl groups and has a viscosity of 200-280 seconds as determined by ASTM Method D 1343. The cellulose acetate grade 2 comprises 3-5% by weight of hydroxyl groups and has a viscosity of 6 to 45 seconds. The cellulose acetate grade 3 comprises 3-5% by weight of hydroxyl groups and has a viscosity of 100 to 240 seconds.

Some exemplary grades of cellulose acetate that are suitable for use in the making the semipermeable membrane are also described in the table below, which is included by way of example. Cellulose acetate of differing grades is readily available from Eastman Chemical Company (Kingsport, Tenn., USA).

| Cellulose | Hydroxyl Content | Acetyl Content | Viscosity* |
|---|---|---|---|
| Acetate | (% by wt.) | (% by wt.) | (seconds) |
| Grade 1 | 7-10 | 30-36 | 200-280 |
| Grade 2 | 3-5 | 37-43 | 6-45 |
| Grade 3 | 3-5 | 37-43 | 100-240 |

*Viscosity determined as set forth in ASTM D817 (Formula A) and D1343, the disclosure of which is hereby incorporated by reference.

Plasticizers can be included in the present device to modify the properties and characteristics of the polymers used in the coats or core of the device. As used herein, the term “plasticizer” includes all compounds capable of plasticizing or softening a polymer or binder used in invention. The plasticizer should be able to lower the melting temperature or glass transition temperature (softening point temperature) of the polymer or binder. Plasticizers, such as low molecular weight PEG, generally broaden the average molecular weight of a polymer in which they are included thereby lowering its glass transition temperature or softening point. Plasticizers also generally reduce the viscosity of a polymer. It is possible the plasticizer will impart some particularly advantageous physical properties to the osmotic device of the invention.

Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. It is also contemplated and within the scope of the invention, that a combination of plasticizers may be used in the present formulation. The PEG based plasticizers are available commercially or can be made by a variety of methods, such as disclosed in Poly(ethylene glycol) Chemistry: Biotechnical and Biomedical Applications (J. M. Harris, Ed.; Plenum Press, NY) the disclosure of which is hereby incorporated by reference.

In one embodiment of the invention the membrane ruptures during use of the osmotic device to form a spaced away second aperture such that the device provides an increased release rate of active agent during use as compared to a control osmotic device which membrane does not rupture, and the passageways together provide a controlled release of the contents of the core. The preformed passageway does not connect with the passageway formed in situ (in the environment of use) by rupture, meaning that the second passageway, after being formed, remains spaced away from the preformed passageway.

The osmotic device of the invention can comprise a water soluble and/or erodible coating, which is inert or which contains drug. This coating would cover and surround the semipermeable membrane and plug any preformed passageway in the membrane if the passageway had been formed prior to addition of the coating. The water soluble and/or erodible coating will generally comprise an inert and non-toxic material that is at least partially, and optionally substantially completely, soluble or erodible in an environment of use. Selection of materials suitable for the inert or drug-containing water soluble coatings will depend upon the desired release rate of drug from the drug-containing coating and upon the desired separation of drug delivery from the core versus the drug-containing coating. A rapidly dissolving coat will be soluble in the buccal cavity and/or upper gastrointestinal (GI) tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the relevant disclosures of which are hereby incorporated by reference. In some embodiments, the rapidly dissolving coat will be soluble in saliva, gastric juices, or acidic fluids.

Materials which are suitable for making the water soluble and/or erodible coatings of the invention include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those of ordinary skill in the art. Other lamina forming materials that can be used for this purpose include poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer. The water soluble coating can comprise other pharmaceutical excipients that do or do not alter the way in which the water soluble coating behaves. The artisan of ordinary skill will recognize that the above-noted materials include film-forming polymers.

Other materials which can be used in the water soluble and/or erodible coatings include hydroxypropylcellulose, microcrystalline cellulose (MCC, Avicel™ from FMC Corp.), poly(ethylene-vinyl acetate) (60:40) copolymer (EVAC from Aldrich Chemical Co.), 2-hydroxyethylmethacrylate (HEMA), MMA, terpolymers of HEMA:MMA:MA synthesized in the presence of N,N′-bis(methacryloyloxyethyloxycarbonylamino)-azobenzene, azopolymers, enteric coated timed release system (Time Clock® from Pharmaceutical Profiles, Ltd., UK) and calcium pectinate can be included in the water soluble coat.

The inert water soluble and/or erodible coat covering the semipermeable wall and blocking the passageway is made of synthetic or natural material that, through selective dissolution or erosion, allows the passageway to become unblocked thus allowing the process of osmotic delivery to start. This slow or fast dissolving water soluble coat can be impermeable to a first external fluid, while being soluble in a second external fluid. This property can help to achieve a controlled and selective release of the active compound in the nucleus.

In some embodiments, the inert water soluble and/or erodible coat will be insoluble in the fluid of a first environment of use, such as gastric juices, acidic fluids, or polar liquids, and soluble or erodible in the fluid of a second environment of use, such as intestinal juices, substantially pH neutral or basic fluids, or apolar liquids. A wide variety of other polymeric materials are known to possess these various solubility properties and can be included in the water soluble coat. Such other polymeric materials include, by way of example and without limitation, cellulose acetate phthalate (CAP), cellulose acetate trimelletate (CAT), poly(vinyl acetate)phthalate (PVAP), hydroxypropyl methylcellulose phthalate (HP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit™ L-30-D (MA-EA, 1:1), Eudragit™ L-100-55 (MA-EA, 1:1), hydroxypropyl methylcellulose acetate succinate (HPMCAS), Coateric™ (PVAP), Aquateric™ (CAP), AQOAT™ (HPMCAS) and combinations thereof. The water-soluble coat can also comprise dissolution aids, stability modifiers, and bioabsorption enhancers.

An optional polymeric material for use in the inert water soluble and/or erodible coat includes enteric materials that resist the action of gastric fluid avoiding permeation through the semipermeable wall while one or more of the materials in the core are solubilized in the intestinal tract thereby allowing delivery of a drug in the core by osmotic pumping to begin. A material that easily adapts to this kind of requirement is a poly(vinylpyrrolidone)-vinyl acetate copolymer, such as the material supplied by BASF under its Kollidon VA64™, mixed with magnesium stearate and other similar excipients. The water soluble and/or erodible coat can also comprise povidone, which is supplied by BASF under its Kollidon K 30™, and hydroxypropyl methylcellulose, which is supplied by Dow under its Methocel E-15™. The materials can be prepared in solutions having different concentrations of polymer according to the desired solution viscosity. For example, a 10% p/v aqueous solution of Kollidon™ K 30 has a viscosity of about 5.5-8.5 cps at 20° C., and a 2% p/v aqueous solution of Methocel™ E-15 has a viscosity of about 13-18 cps at 20° C.

The inert water soluble and/or erodible coat can also comprise other materials suitable which are substantially resistant to gastric juices and which will promote either enteric or colonic release. For this purpose, the inert water soluble and/or erodible coat can comprise one or more materials that do not dissolve, disintegrate, or change their structure in the stomach and during the period of time that the osmotic device resides in the stomach. Representative materials that keep their integrity in the stomach can comprise a member selected from the group consisting of (a) keratin, keratin sandarac-tolu, salol (phenyl salicylate), salol beta-naphthylbenzoate and acetotannin, salol with balsam of Peru, salol with tolu, salol with gum mastic, salol and stearic acid, and salol and shellac; (b) a member selected from the group consisting of formalized protein, formalized gelatin, and formalized cross-linked gelatin and exchange resins; (c) a member selected from the group consisting of myristic acid-hydrogenated castor oil-cholesterol, stearic acid-mutton tallow, stearic acid-balsam of tolu, and stearic acid-castor oil; (d) a member selected from the group consisting of shellac, ammoniated shellac, ammoniated shellac-salol, shellac-wool fat, shellac-acetyl alcohol, shellac-stearic acid-balsam of tolu, and shellac n-butyl stearate; (e) a member selected from the group consisting of abietic acid, methyl abietate, benzoin, balsam of tolu, sandarac, mastic with tolu, and mastic with acetyl alcohol; (f) acrylic resins represented by anionic polymers synthesized from methacrylic acid and methacrylic acid methyl ester, copolymeric acrylic resins of methacrylic and methacrylic acid and methacrylic acid alkyl esters, copolymers of alkacrylic acid and alkacrylic acid alkyl esters, acrylic resins such as dimethylaminoethylmethacrylate-butylmethacrylate-methylmethacrylate copolymer of 150,000 molecular weight, methacrylic acid-methylmethacrylate 50:50 copolymer of 135,000 molecular weight, methacrylic acid-methylmethacrylate 30:70-copolymer of 135,000 mol. wt., methacrylic acid-dimethylaminoethyl-methacrylate-ethylacrylate copolymer of 750,000 mol. wt., methacrylic acid-methylmethacrylate-ethylacrylate copolymer of 1,000,000 mol. wt., and ethylacrylate-methylmethacrylate-ethylacrylate copolymer of 550,000 mol. wt; and, (g) an enteric composition comprising a member selected from the group consisting of cellulose acetyl phthalate, cellulose diacetyl phthalate, cellulose triacetyl phthalate, cellulose acetate phthalate, hydroxypropyl methylcellulose phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate wherein the alkyl comprises from 1 to 7 straight and branched alkyl groups, aryl phthalates, and other materials known to one or ordinary skill in the art.

An alternative embodiment of the invention includes pore former(s) in the wall to form additional passageways over time.

Release of active agent from the core can be delayed such that the release profile of active agent will exhibit delayed and then controlled release. Such a device would be termed a delayed controlled release device.

The osmotic device of the invention comprises at least one passageway (pore, hole, or aperture) that communicates the exterior of the semipermeable wall with the core of the device. The passageway can be formed according to any of the known methods of forming passageways in a semipermeable membrane. Such methods include, for example, 1) drilling a hole through the semipermeable membrane with a bit or laser; 2) including a water soluble material within the composition that forms the semipermeable membrane such that a pore forms when the osmotic device is in an aqueous environment of use; 3) punching a hole through the semipermeable membrane; or 4) employing a tablet punch having a pin to punch a hole through the semipermeable lamina. The passageway can pass through the semipermeable wall and one or more of any other lamina coated onto the semipermeable membrane or between the semipermeable membrane and the core. The passageway(s) can be shaped as desired. In some embodiments, the passageway is laser drilled and is shaped as an oval, ellipse, slot, slit, cross or circle.

Methods of forming passageways in semipermeable membranes of osmotic devices are disclosed in U.S. Pat. No. 4,088,864 to Theeuwes et al., U.S. Pat. No. 4,016,880 to Theeuwes et al., U.S. Pat. No. 3,916,899 to Theeuwes et al., U.S. Pat. No. 4,285,987 to Ayer et al., U.S. Pat. No. 4,783,337 to Wong et al., U.S. Pat. No. 5,558,879 to Chen et al., U.S. Pat. No. 4,801,461 to Hamel et al., U.S. Pat. No. 3,845,770 to Theeuwes et al., PCT International Publication No. WO 04/103349 to Faour, and U.S. Pat. No. 6,809,288 to Faour, the disclosures of which are hereby incorporated by reference.

The preformed passageway in the wall is typically generated by mechanical means, such as perforation by a laser or drill, or any other similar method known to those of ordinary skill in the art. The passageway is generally formed by controlled laser perforation, using an apparatus similar to that disclosed in Theeuwes et al. '864, the entire disclosure of which is incorporated herein by reference. Specific embodiments of the controlled laser perforation method will vary according to the equipment used. The laser equipment of Theeuwes et al. '864 can be modified as described herein to prepare an osmotic device according to the invention. Other suitable laser equipment, and methods of use thereof, are disclosed in Emerton et al. '793 and Roy '771, the entire disclosures of which are hereby incorporated by reference. The process and system of Faour (U.S. Pregrant Patent Publication No. 2002/0099361) can also be used to form the preformed passageway and/or etch in the wall.

A preformed passageway can be made to substantially retain its size during use of the device or it can be made to increase in size during use of the dosage form. Preformed passageways of different sizes, shapes and functions can be used.

In one embodiment of the invention the preformed passageway in the wall may dissolve or tear in a predetermined or random manner, and the shape of the preformed passageway after enlargement can be made to approximate a predetermined or randomly determined shape. The extent to which a passageway increases in size can also be related to the viscosity, molecular weight or degree of substitution of the at least one excipient. Generally, increasing the viscosity, molecular weight, or degree of substitution of the at least one excipient will increase the extent to which the passageway increases in size.

A device according to the present invention can comprise one or more preformed passageways including two, three, four, five, six, seven, eight, nine, ten or more preformed passageways. It is only necessary that the preformed passageways together are adapted to permit controlled release of ingredients from the core during use. In some embodiments, the membrane comprises one preformed passageway having a diameter ranging from 0.2 mm to 0.8 mm. In other embodiments, the total area of the preformed passageway(s) present in the membrane ranges from 0.12 mm² to 2.1 mm².

The osmotic device of the invention can also comprise an adsorbent, antioxidant, buffering agent, colorant, flavorant, sweetening agent, antiadherent, binder, diluent, direct compression excipient, disintegrant, glidant, lubricant, opaquant and/or polishing agent.

As used herein, the term “adsorbent” is intended to mean an agent capable of holding other molecules onto its surface by physical or chemical (chemisorption) means. Such compounds include, by way of example and without limitation, powdered and activated charcoal and other materials known to one of ordinary skill in the art.

As used herein, the term “antioxidant” is intended to mean an agent that inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate and sodium metabisulfite and other materials known to one of ordinary skill in the art.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and other materials known to one of ordinary skill in the art.

As used herein, the term “antiadherent” is intended to mean an agent that prevents the sticking of tablet formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

As used herein, the term “binder” is intended to mean a substance used to cause adhesion of powder particles in granulations. Such compounds include, by way of example and without limitation, acacia, poly(vinylpyrrolidone), compressible sugar (e.g., NuTab™), ethylcellulose, gelatin, liquid glucose, povidone, pregelatinized starch, tragacanth, starch, cellulose materials such as methyl cellulose and sodium carboxy methyl cellulose, alginic acids and salts thereof, polyethylene glycol, guar gum, polysaccharides, bentonites, sugars, invert sugars, poloxamers (PLURONIC™ F68, PLURONIC™ F127), collagen, albumin, cellulosics in nonaqueous solvents, combinations thereof and the like.

Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

As used herein, the term “diluent” or “filler” is intended to mean an inert substance used as filler to create the desired bulk, flow properties, and compression characteristics in the preparation of tablets and capsules. Such compounds include, by way of example and without limitation, dibasic calcium phosphate, kaolin, lactose, sucrose, mannitol, microcrystalline cellulose, powdered cellulose, precipitated calcium carbonate, sorbitol, and starch and other materials known to one of ordinary skill in the art.

As used herein, the term “direct compression excipient” is intended to mean a compound used in direct compression tablet formulations. Such compounds include, by way of example and without limitation, dibasic calcium phosphate (e.g., Ditab) and other materials known to one of ordinary skill in the art.

As used herein, the term “glidant” is intended to mean agents used in tablet and capsule formulations to promote the flowability of a granulation. Such compounds include, by way of example and without limitation, colloidal silica, cornstarch, talc, calcium silicate, magnesium silicate, colloidal silicon, silicon hydrogel and other materials known to one of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, and zinc stearate and other materials known to one of ordinary skill in the art.

As used herein, the term “opaquant” is intended to mean a compound used to render a capsule or a tablet coating opaque. May be used alone or in combination with a colorant. Such compounds include, by way of example and without limitation, titanium dioxide and other materials known to one of ordinary skill in the art.

As used herein, the term “polishing agent” is intended to mean a compound used to impart an attractive sheen to coated tablets. Such compounds include, by way of example and without limitation, carnauba wax, and white wax and other materials known to one of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, crospovidone (cross linked polyvinyl pyrrolidone), sweeteners, clays, such as bentonite, microcrystalline cellulose (e.g., Avicel), carboxymethylcellulose calcium, cellulose polyacrilin potassium (e.g., Amberlite), alginates, sodium starch glycolate, gums such as agar, guar, locust bean, karaya, pectin, tragacanth and other materials known to one of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide red, other F.D. & C. dyes and natural coloring agents such as grape skin extract, beet red powder, beta-carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particularly preferred flavors are the grape and cherry flavors and citrus flavors such as orange.

The present device can also employ one or more commonly known surface active agents or cosolvents that improve wetting or disintegration of the osmotic device core or layers.

It is contemplated that the osmotic device of the invention can also include oils, for example, fixed oils, such as peanut oil, sesame oil, cottonseed oil, corn oil and olive oil; fatty acids, such as oleic acid, stearic acid and isotearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for detergent compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl aminopropionates and 2-alkylimidazoline quaternary ammonium salts; and mixtures thereof.

Various other components, not otherwise listed above, can be added to the present formulation for optimization of a desired active agent release profile including, by way of example and without limitation, glycerylmonostearate, nylon, cellulose acetate butyrate, d,l-poly(lactic acid), 1,6-hexanediamine, diethylenetriamine, starches, derivatized starches, acetylated monoglycerides, gelatin coacervates, poly(styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

It should be understood, that compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A water insoluble active agent such as lercanidipine is primarily released as insoluble particles, which therefore have limited bioavailability. The concentration of dissolved lercanidipine may be temporarily improved sufficiently to improve absorption through one or more of the following methods: a) delivery of solubilizers such as surfactants, citrate esters, and organic acids, b) increasing dissolution rate by utilizing lercanidipine that have a reduced particle size, c) by co-delivery of a concentration-enhancing polymer, or d) combinations thereof. Examples of the surfactants include non-ionic and/or anionic surfactants, such as Tween 20, Tween 60 or Tween 80, polyoxyethylene or polyethylene-containing surfactants, or other long chain anionic surfactants, particularly sodium lauryl sulfate. Examples of citrate ester derivatives are the alkyl esters, such as triethyl citrate. The use of a highly soluble organic acid as solubilizer serves multiple purposes: it improves the solubility of lercanidipine, particularly when the use environment is at a pH above about 5 to 6; it provides an osmotic pressure differential; it makes the lercanidipine-containing composition more hydrophilic so that it readily wets; and it acts as a fluidizing agent, lowering the viscosity of the lercanidipine-containing composition rapidly. Examples of organic acid solubilizers include adipic acid, citric acid, fumaric acid, tartaric acid, succinic acid, and the like.

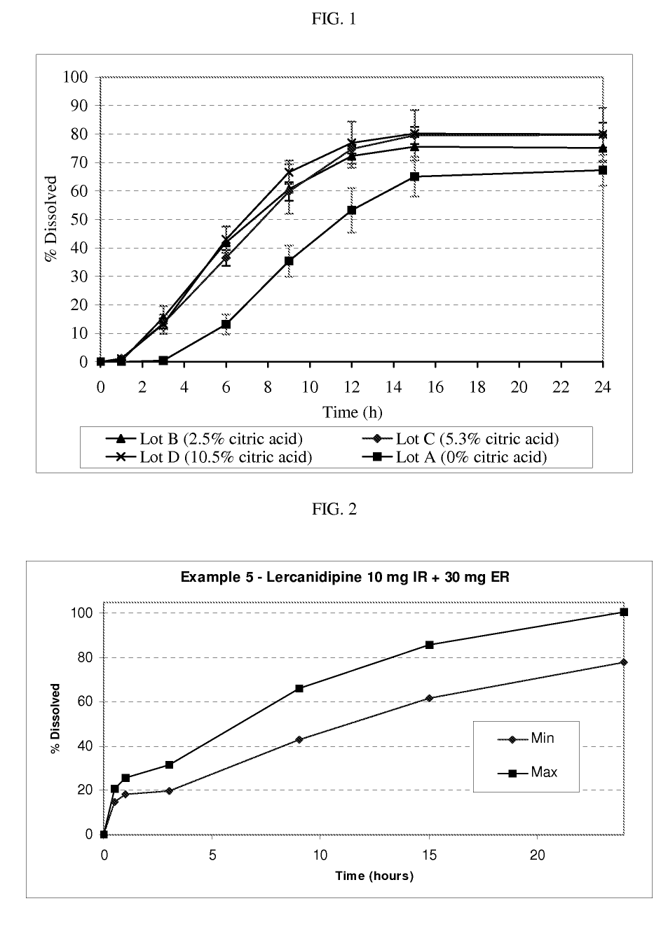

Example 1 discloses four lercanidipine II osmotic device formulations differing only in the amount of citric acid, lots A, B, C, and D contain 0%, 2.5%, 5.3%, and 10.5% of citric acid respectively. The lercanidipine is present as the hydrochloride salt Form II polymorph. FIG. 1 shows that the osmotic device formulations containing citric acid provide a faster and higher release amount of lercanidipine (lots B, C, and D) than the osmotic device without citric acid (lot A). The in vitro testing for lots A, B, C, and D was performed with USP Type II dissolution apparatus (paddles), in 900 ml of water with 0.3% polysorbate 80, with a fixed agitation rate of 100 revolutions per minute, maintained at a temperature of 37±0.5° C. The samples were tested by high pressure liquid chromatography.

The release profiles obtained for six tablets (#1-#6) of the formulation containing 0% of citric acid (lot A) are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 0.0 | 0.0 | 0.0 | 0.0 | 0.0 | 0.0 | 0.0 | 0.0 | 0.0 | 0.0 |
| 3 | 1.0 | 0.0 | 0.0 | 0.6 | 0.9 | 0.0 | 0.4 | 0.5 | 0.0 | 1.0 |
| 6 | 19.6 | 12.2 | 12.3 | 10.9 | 14.4 | 9.4 | 13.1 | 3.6 | 9.4 | 19.6 |
| 9 | 41.1 | 39.2 | 33.1 | 31.3 | 39.8 | 27.7 | 35.4 | 5.4 | 27.7 | 41.1 |
| 12 | 55.0 | 60.0 | 46.7 | 49.6 | 63.8 | 44.1 | 53.2 | 7.8 | 44.1 | 63.8 |
| 15 | 65.1 | 69.2 | 61.0 | 60.1 | 76.7 | 57.9 | 65.0 | 7.0 | 57.9 | 76.7 |
| 24 | 67.3 | 67.3 | 62.1 | 66.8 | 77.3 | 62.9 | 67.3 | 5.4 | 62.1 | 77.3 |

The release profiles obtained for six tablets (#1-#6) of the formulation containing 2.5% of citric acid (lot B) are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 0.2 | 0.1 | 0.1 | 0.2 | 0.3 | 1.1 | 0.3 | 0.4 | 0.1 | 1.1 |
| 3 | 15.2 | 8.1 | 14.8 | 17.3 | 18.5 | 19.3 | 15.5 | 4.0 | 8.1 | 19.3 |
| 6 | 42.1 | 32.7 | 48.9 | 39.3 | 42.3 | 46.1 | 41.9 | 5.6 | 32.7 | 48.9 |
| 9 | 66.5 | 52.8 | 71.7 | 51.3 | 55.1 | 66.7 | 60.7 | 8.6 | 51.3 | 71.7 |
| 12 | 77.7 | 69.1 | 76.8 | 69.1 | 68.2 | 72.6 | 72.2 | 4.1 | 68.2 | 77.7 |
| 15 | 82.9 | 75.3 | 79.5 | 72.1 | 70.7 | 72.7 | 75.5 | 4.8 | 70.7 | 82.9 |
| 24 | 82.8 | 80.0 | 71.1 | 72.2 | 70.6 | 74.0 | 75.1 | 5.1 | 70.6 | 82.8 |

The release profiles obtained for six tablets (#1-#6) of the formulation containing 5.3% of citric acid (lot C) are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 2.0 | 0.5 | 1.4 | 1.0 | 1.3 | 1.0 | 1.2 | 0.5 | 0.5 | 2.0 |
| 3 | 15.3 | 11.6 | 11.0 | 14.0 | 13.9 | 15.1 | 13.5 | 1.8 | 11.0 | 15.3 |
| 6 | 35.5 | 37.1 | 34.1 | 33.4 | 38.3 | 40.6 | 36.5 | 2.7 | 33.4 | 40.6 |
| 9 | 61.3 | 60.5 | 57.2 | 54.6 | 62.2 | 63.2 | 59.8 | 3.3 | 54.6 | 63.2 |
| 12 | 75.7 | 75.5 | 74.6 | 71.7 | 74.7 | 76.1 | 74.7 | 1.6 | 71.7 | 76.1 |
| 15 | 84.1 | 80.2 | 77.6 | 78.5 | 75.4 | 81.3 | 79.5 | 3.0 | 75.4 | 84.1 |
| 24 | 85.3 | 79.5 | 76.9 | 81.5 | 72.7 | 82.0 | 79.6 | 4.4 | 72.7 | 85.3 |

The release profiles obtained for six tablets (#1-#6) of the formulation containing 10% of citric acid (lot D) are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 1.7 | 1.8 | 1.1 | 0.5 | 0.9 | 0.7 | 1.1 | 0.5 | 0.5 | 1.8 |
| 3 | 13.4 | 18.9 | 13.0 | 8.4 | 14.0 | 12.0 | 13.3 | 3.4 | 8.4 | 18.9 |
| 6 | 41.7 | 50.0 | 44.0 | 36.1 | 40.6 | 45.2 | 43.0 | 4.7 | 36.1 | 50.0 |
| 9 | 69.5 | 70.8 | 65.6 | 64.1 | 60.0 | 69.3 | 66.6 | 4.1 | 60.0 | 70.8 |
| 12 | 87.4 | 77.6 | 78.2 | 74.0 | 64.8 | 79.4 | 76.9 | 7.4 | 64.8 | 87.4 |
| 15 | 88.3 | 81.7 | 86.4 | 78.5 | 65.1 | 81.1 | 80.2 | 8.2 | 65.1 | 88.3 |
| 24 | 89.6 | 80.4 | 86.4 | 81.3 | 62.9 | 78.9 | 79.9 | 9.3 | 62.9 | 89.6 |

Tartaric acid has a significant destabilizing effect on the stability of lercanidipine hydrochloride as shown in Example 2 by the high amount of impurities generated (5.83% at 50° C. and 75% RH). Citric acid has very mild destabilizing effect on the stability of lercanidipine hydrochloride (1.24% at 50° C. and 75% RH) and it provides a faster and higher release amount of lercanidipine than an osmotic device without citric acid, as shown in FIG. 1. The results indicate that citric acid is a performance-enhancing acid, since it enhances the in vitro dissolution of lercanidipine as compared to tartaric acid and it has a much lower destabilizing effect on lercanidipine than does tartaric acid.

The performance-enhancing acid can be present in different amounts in the controlled release dosage form of the invention. It may provide different levels of performance enhancement, of the controlled release dosage form, depending upon the amount that is included in the dosage form, e.g. the core of the dosage form. The performance-enhancing acid can be present in the core in an amount of greater than 0% wt. up to about 2.5% wt., 5% wt., 10% wt. or 15% wt. based upon the weight of the uncoated core. Accordingly, the weight ratio of lercanidipine to performance-enhancing acid in the core may vary and can be optimized to provide the desired level of enhancement in performance of the dosage form.

The predicted release profiles of the osmotic device formulations containing lercanidipine (30 mg strength) in the core and lercanidipine (10 mg strength) in an immediate or rapid release external drug-containing coat of Example 5 are disclosed in the table below.

| | |
|---|---|
| Time | Range (%) |

| (hrs) | Max | Min |
|---|---|---|
| 0 | 0 | 0 |
| 0.5 | 15 | 21 |
| 1 | 18 | 26 |
| 3 | 20 | 31 |
| 9 | 43 | 66 |
| 15 | 62 | 86 |
| 24 | 78 | 100 |

FIG. 2 shows the predicted release profiles of the osmotic device formulations containing lercanidipine (30 mg strength) in the core and lercanidipine (10 mg strength) in an immediate or rapid release external drug-containing coat disclosed in Example 5.

The predicted release profiles of the osmotic device formulations containing lercanidipine (50 mg strength) in the core and lercanidipine (10 mg strength) in an immediate or rapid release external drug-containing coat of Example 5 are disclosed in the table below.

| | |
|---|---|
| Time | Range (%) |

| (hrs) | Min | Max |
|---|---|---|
| 0 | 0 | 0 |
| 0.5 | 10 | 14 |
| 1 | 12 | 18 |
| 3 | 14 | 24 |
| 9 | 40 | 63 |
| 15 | 60 | 84 |
| 24 | 79 | 100 |

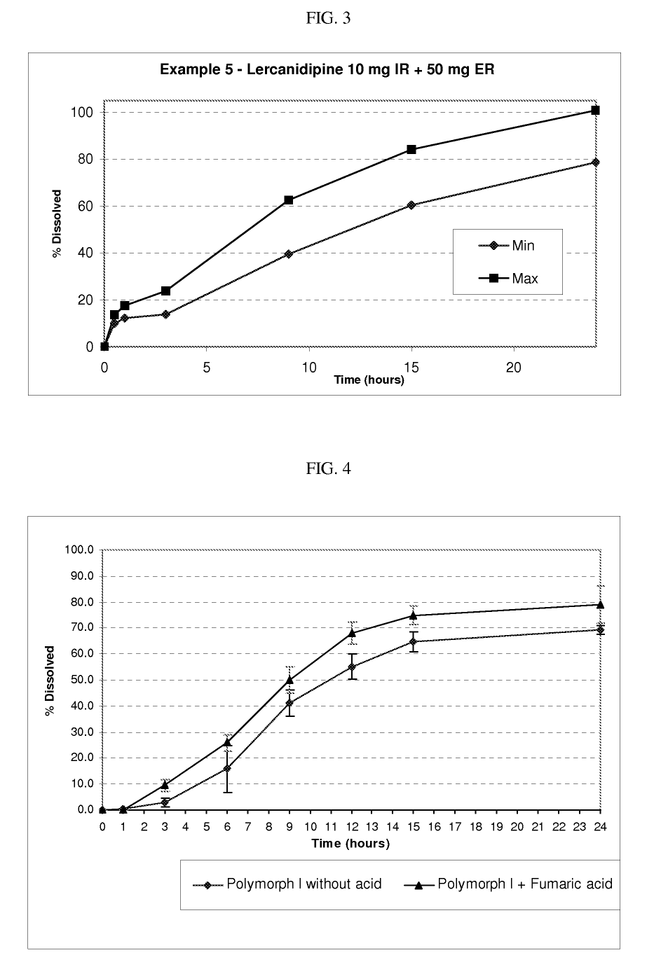

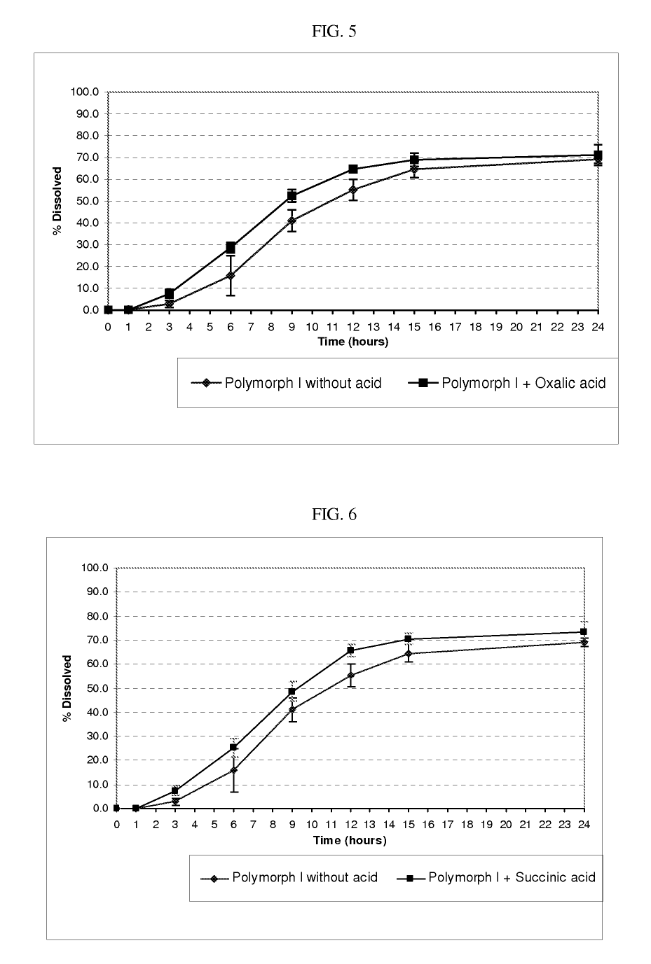

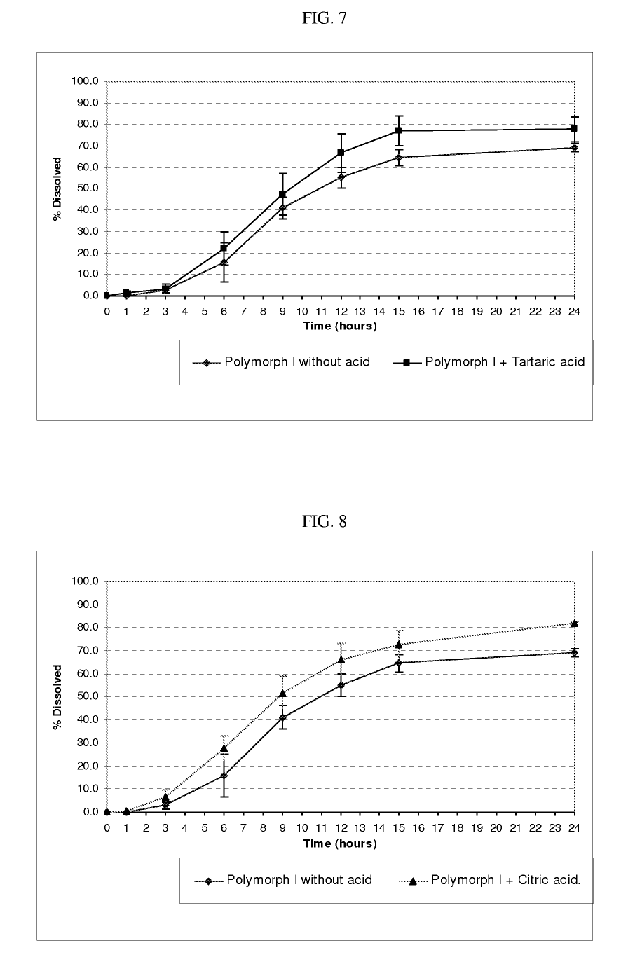

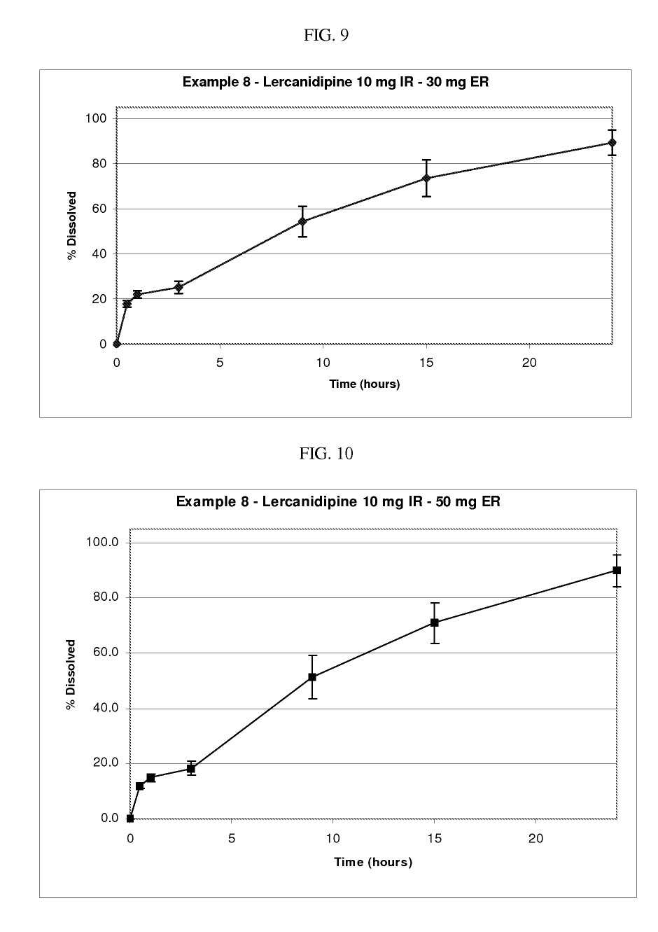

FIG. 3 shows the predicted release profiles of the osmotic device formulations containing lercanidipine (50 mg strength) in the core and lercanidipine (10 mg strength) in an immediate or rapid release external drug-containing coat disclosed in Example 5.

Example 6 discloses lercanidipine I osmotic device formulations differing only in the acidifying agent used. The in vitro testing was performed with USP Type II dissolution apparatus (paddles), in 900 ml of water with 0.3% polysorbate 80, with a fixed agitation rate of 100 revolutions per minute, maintained at a temperature of 37±0.5° C. The samples were tested by high pressure liquid chromatography.

The release profiles obtained for four tablets (#1-#4) of the formulation without acidifying agent are disclosed in the table below, which detail the amount of lercanidipine released at the indicated time points, based upon when the osmotic device was exposed to the release liquid medium.

| Time | Released % | | | | Mean | SD | Range (%) | |
|---|---|---|---|---|---|---|---|---|
| (hrs) | #1 | #2 | #3 | #4 | (%) | (%) | Min | Max |
| 0 | 0.0 | 0.0 | 0.0 | 0.0 | 0.0 | 0.0 | 0.0 | 0.0 |
| 1 | 0.2 | 0.2 | 0.2 | 0.2 | 0.2 | 0.0 | 0.2 | 0.2 |
| 3 | 2.9 | 3.6 | 0.6 | 4.4 | 2.9 | 1.6 | 0.6 | 4.4 |
| 6 | 17.6 | 24.3 | 2.8 | 18.5 | 15.8 | 9.1 | 2.8 | 24.3 |
| 9 | 37.7 | 46.7 | 43.7 | 36.1 | 41.0 | 5.0 | 36.1 | 46.7 |
| 12 | 53.0 | 59.2 | 59.2 | 49.5 | 55.2 | 4.8 | 49.5 | 59.2 |
| 15 | 64.8 | 67.0 | 67.4 | 59.1 | 64.6 | 3.8 | 59.1 | 67.4 |
| 24 | 71.1 | 70.1 | 68.1 | 67.4 | 69.2 | 1.7 | 67.4 | 71.1 |

The release profiles obtained for six tablets (#1-#6) of the formulation containing fumaric acid as acidifying agent are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 0.0 | 0.0 | 0.0 | 0.0 | 0.0 | 0.0 | 0.0 | 0.0 | 0.0 | 0.0 |
| 3 | 13.2 | 9.6 | 8.3 | 6.7 | 10.5 | 8.4 | 9.5 | 2.2 | 6.7 | 13.2 |
| 6 | 29.0 | 24.3 | 24.5 | 25.2 | 22.1 | 30.0 | 25.9 | 3.0 | 22.1 | 30.0 |
| 9 | 53.3 | 50.9 | 43.8 | 51.8 | 43.8 | 55.7 | 49.9 | 5.0 | 43.8 | 55.7 |
| 12 | 71.1 | 65.9 | 62.5 | 69.3 | 65.2 | 74.2 | 68.0 | 4.3 | 62.5 | 74.2 |
| 15 | 74.4 | 74.6 | 71.2 | 72.0 | 76.4 | 81.1 | 75.0 | 3.5 | 71.2 | 81.1 |
| 24 | 72.9 | 84.6 | 76.0 | 72.7 | 77.3 | 90.6 | 79.0 | 7.1 | 72.7 | 90.6 |

The release profiles obtained for six tablets (#1-#6) of the formulation containing oxalic acid as acidifying agent are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 0.1 | 0.1 | 0.1 | 0.1 | 0.1 | 0.1 | 0.1 | 0.0 | 0.1 | 0.1 |
| 3 | 10.6 | 7.8 | 8.4 | 5.5 | 5.0 | 8.1 | 7.6 | 2.1 | 5.0 | 10.6 |
| 6 | 32.1 | 25.5 | 29.1 | 27.5 | 27.1 | 30.8 | 28.7 | 2.5 | 25.5 | 32.1 |
| 9 | 56.9 | 52.0 | 50.7 | 48.8 | 51.5 | 54.4 | 52.4 | 2.9 | 48.8 | 56.9 |
| 12 | 67.8 | 65.2 | 64.2 | 62.9 | 63.4 | 64.6 | 64.7 | 1.7 | 62.9 | 67.8 |
| 15 | 68.3 | 73.8 | 71.0 | 69.1 | 65.3 | 66.4 | 69.0 | 3.1 | 65.3 | 73.8 |
| 24 | 68.5 | 75.1 | 77.7 | 72.9 | 65.6 | 67.6 | 71.2 | 4.7 | 65.6 | 77.7 |

The release profiles obtained for six tablets (#1-#6) of the formulation containing succinic acid as acidifying agent are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 1 | 0.1 | 0.0 | 0.0 | 0.0 | 0.0 | 0.0 | 0.0 | 0.0 | 0.0 | 0.1 |
| 3 | 11.1 | 7.0 | 7.2 | 7.5 | 7.0 | 5.0 | 7.5 | 2.0 | 5.0 | 11.1 |
| 6 | 28.9 | 29.1 | 24.7 | 26.3 | 23.2 | 18.9 | 25.2 | 3.8 | 18.9 | 29.1 |
| 9 | 54.9 | 49.8 | 45.9 | 51.3 | 46.3 | 43.5 | 48.6 | 4.2 | 43.5 | 54.9 |
| 12 | 68.7 | 66.7 | 64.2 | 68.4 | 62.8 | 63.4 | 65.7 | 2.6 | 62.8 | 68.7 |
| 15 | 75.0 | 71.5 | 69.1 | 69.7 | 69.5 | 68.9 | 70.6 | 2.3 | 68.9 | 75.0 |
| 24 | 80.4 | 73.5 | 73.5 | 69.9 | 74.7 | 69.3 | 73.5 | 4.0 | 69.3 | 80.4 |

The release profiles obtained for four tablets (#1-#4) of the formulation containing tartaric acid as acidifying agent are disclosed in the table below, which detail the amount of lercanidipine released at the indicated time points, based upon when the osmotic device was exposed to the release liquid medium.

| Time | Released % | | | | Mean | SD | Range (%) | |
|---|---|---|---|---|---|---|---|---|
| (hrs) | #1 | #2 | #3 | #4 | (%) | (%) | Min | Max |
| 0 | 0.0 | 0.0 | 0.0 | 0.0 | 0.0 | 0.0 | 0.0 | 0.0 |
| 1 | 2.2 | 1.9 | 0.8 | 1.2 | 1.5 | 0.6 | 0.8 | 2.2 |
| 3 | 5.7 | 0.5 | 3.8 | 3.5 | 3.4 | 2.1 | 0.5 | 5.7 |
| 6 | 30.8 | 11.9 | 21.5 | 24.4 | 22.1 | 7.9 | 11.9 | 30.8 |
| 9 | 56.6 | 34.5 | 46.1 | 53.0 | 47.5 | 9.8 | 34.5 | 56.6 |
| 12 | 74.5 | 55.1 | 64.0 | 73.1 | 66.7 | 9.0 | 55.1 | 74.5 |
| 15 | 83.4 | 67.9 | 76.2 | 80.6 | 77.0 | 6.8 | 67.9 | 83.4 |
| 24 | 84.8 | 71.0 | 75.7 | 79.4 | 77.7 | 5.8 | 71.0 | 84.8 |

The release profiles obtained for four tablets (#1-#4) of the formulation containing citric acid as acidifying agent are disclosed in the table below, which detail the amount of lercanidipine released at the indicated time points, based upon when the osmotic device was exposed to the release liquid medium.

| Time | Released % | | | | Mean | SD | Range (%) | |
|---|---|---|---|---|---|---|---|---|
| (hrs) | #1 | #2 | #3 | #4 | (%) | (%) | Min | Max |
| 0 | 0.0 | 0.0 | 0.0 | 0.0 | 0.0 | 0.0 | 0.0 | 0.0 |
| 1 | 0.4 | 0.6 | 0.4 | 0.4 | 0.5 | 0.1 | 0.4 | 0.6 |
| 3 | 5.5 | 3.4 | 8.6 | 9.6 | 6.8 | 2.8 | 3.4 | 9.6 |
| 6 | 22.6 | 24.9 | 29.5 | 34.4 | 27.8 | 5.2 | 22.6 | 34.4 |
| 9 | 42.9 | 49.1 | 54.7 | 59.7 | 51.6 | 7.3 | 42.9 | 59.7 |
| 12 | 57.9 | 63.7 | 69.9 | 73.4 | 66.2 | 6.8 | 57.9 | 73.4 |
| 15 | 64.6 | 72.8 | 74.6 | 79.1 | 72.8 | 6.1 | 64.6 | 79.1 |
| 24 | 82.1 | 82.6 | 81.3 | 81.0 | 81.8 | 0.7 | 81.0 | 82.6 |

Fumaric acid, oxalic acid and succinic acid do not have a destabilizing effect on the stability of lercanidipine hydrochloride as shown in Example 2 by the low amount of impurities generated (0.18%, 0.21% and 0.21% respectively at 50° C. and 75% RH). The results indicate that fumaric acid, oxalic acid and succinic acid, which are dicarboxylic acids, are performance-enhancing acids, since they enhance the in vitro dissolution of lercanidipine as compared to a formulation excluding the performance-enhancing acid and they exhibit reduced degradation of lercanidipine, as compared to a similar formulation containing ascorbic acid, citric acid, tartaric acid or malic acid which are alpha hydroxy carboxylic acids.

The expected values after single dose or at steady-state, as described in Example 7, for an osmotic tablet following once daily oral administration, are as follows 1) the mean value of the maximum plasma concentration (Cmax) of lercanidipine is ≦8 ng/mL, preferably ≦6 ng/mL; 2) the mean value of minimum concentration (Cmin) of lercanidipine is ≧0.5 ng/mL in a dosage interval, preferably ≧1.5 ng/mL; and 3) the lercanidipine plasma concentrations is >1 ng/mL at least over 12 hours in a dosage interval, preferably >2 ng/mL.

Example 8 discloses two lercanidipine I osmotic device formulations containing lercanidipine (30 and 50 mg strength) in the core and lercanidipine (10 mg strength) in an immediate or rapid release external drug-containing coat.

The release profiles obtained for six tablets (#1-#6) of the lercanidipine formulation (30 mg strength) in the core and lercanidipine (10 mg strength) in an immediate or rapid release external drug-containing coat of Example 8 are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0 | 0 | 0 | 0 |
| 0.5 | 16.0 | 17.0 | 19.5 | 16.5 | 20.0 | 17.8 | 18 | 1.5 | 16 | 20 |
| 1 | 19.3 | 21.4 | 21.8 | 21.9 | 25.4 | 22.0 | 22 | 1.6 | 19 | 25 |
| 3 | 19.6 | 24.4 | 23.1 | 27.4 | 30.2 | 26.0 | 25 | 2.8 | 20 | 30 |
| 9 | 44.5 | 51.8 | 47.8 | 61.0 | 64.2 | 56.8 | 54 | 6.7 | 45 | 64 |
| 15 | 65.2 | 69.8 | 66.5 | 78.3 | 87.4 | 74.0 | 74 | 8.1 | 65 | 87 |
| 24 | 78.5 | 88.5 | 84.5 | 93.3 | 99.6 | 91.3 | 89 | 5.6 | 79 | 100 |

The release profiles obtained for six tablets (#1-#6) of the lercanidipine formulation (50 mg strength) in the core and lercanidipine (10 mg strength) in an immediate or rapid release external drug-containing coat of Example 8 are disclosed in the table below, which detail the amount of lercanidipine released at the indicated time points, based upon when the osmotic device was exposed to the release liquid medium.

| | Released % | | | | | | | | Range (%) | |
|---|---|---|---|---|---|---|---|---|---|---|
| Time (hrs) | #1 | #2 | #3 | #4 | #5 | #6 | Mean (%) | SD (%) | Min | Max |
| 0 | 0.0 | 0.0 | 0.0 | 0.0 | 0.0 | 0.0 | 0.0 | 0.0 | 0.0 | 0.0 |
| 0.5 | 11.0 | 11.3 | 13.0 | 11.5 | 13.4 | 11.8 | 12.0 | 0.9 | 11.0 | 13.4 |
| 1 | 12.9 | 14.4 | 14.7 | 14.8 | 17.5 | 14.8 | 14.9 | 1.3 | 12.9 | 17.5 |
| 3 | 13.1 | 17.8 | 16.1 | 21.0 | 22.8 | 19.3 | 18.3 | 2.6 | 13.1 | 22.8 |
| 9 | 41.8 | 48.2 | 43.5 | 58.3 | 63.5 | 53.5 | 51.5 | 7.9 | 41.8 | 63.5 |
| 15 | 60.1 | 68.2 | 64.3 | 77.5 | 82.5 | 72.7 | 70.9 | 7.2 | 60.1 | 82.5 |
| 24 | 79.8 | 89.0 | 84.3 | 94.2 | 99.8 | 91.8 | 89.8 | 5.8 | 79.8 | 99.8 |

One or more of the following drugs can be used in combination with lercanidipine in the dosage form: 1) a drug selected from the group consisting of an angiotensin converting enzyme inhibitor, an angiotensin II receptor blocker, a β-blocker, an α-blocker, a diuretic, and mixtures thereof; 2) an angiotensin converting enzyme inhibitor selected from the group consisting of enalapril, captopril, lisinopril, benazepril, enalaprilat, espirapril, fosinopril, moexipril, quinapril, ramipril, perindopril, and trandolapril; 3) an angiotensin II receptor blocker selected from the group consisting of olmesartan, irbesartan, valsartan, telmisartan, losartan and eprosartan; 4) a β-blocker selected from the group consisting of carvedilol, pindolol, propranolol, practolol, metoprolol, esmolol, oxprenolol, timolol, atenolol, alprenolol, sotalol, carteolol, nadolol, betaxolol, penbutolol, acebutolol, and bisoprolol; 5) an α-blocker selected from the group consisting of doxazosin, prazosin, terazosin, and labetalol; 6) enalapril maleate; 7) enalapril maleate and hydrochlorothiazide; 8) lisinopril; 9) lisinopril and hydrochlorothiazide; 10) olmersartan; 11) olmersartan and hydrochlorothiazide; 12) irbesartan; 13) irbesartan and hydrochlorothiazide; 14) carvedilol; 15) carvedilol and hydrochlorothiazide; 16) doxazosin; 17) doxazosin and hydrochlorothiazide; and 18) a diuretic selected from the group consisting of chlorothiazide, acetazolamide, methazolamide, triamterene, furosemide, indapamide, flumethiazide, bumetanide, ethacrynic acid, torsemide, muzolimide, azosemide, piretanide, tripamide, hydrochlorothiazide, chlorthalidone, indapamide, metozalone, cyclopenthiazide, amiloride, xipamide, mefruside, dorzolamide, ethoxzolamide, cyclothiazide, clopamide, dichlorphenamide, hydroflumethiazide, trichlormethiazide, polythiazide and benzothiazide. Such drug combinations can be used to treat a disease or disorder that is therapeutically responsive to lercanidipine and/or the other drug(s) included in the dosage form.

Unless otherwise specified, the term lercanidipine is taken to mean the free base or salt form thereof or a combination thereof. It can be present in the hydrate, solvate, anhydrous or oil form or a combination thereof. Lercanidipine can be present in amorphous or crystalline form or a combination thereof. In crystalline form, any polymorph or combination of two or more different polymorphs of lercanidipine can be present. Lercanidipine can be present in racemic, optically enriched (for either the R or S enantiomer), optically pure form. Combinations of these various forms of lercanidipine can be employed in the controlled release dosage form, osmotic device, of the invention.

The following examples should not be considered exhaustive, but merely illustrative of only a few of the many embodiments contemplated by the present invention. The methods described herein can be followed to prepare osmotic devices according to the invention.

### EXAMPLE 1

Lercanidipine HCl polymorph II osmotic device tablets of 30 mg strengths were manufactured as described herein. The osmotic device tablets contain the following ingredients in the amounts indicated.

| Excipients | Lot A | Lot B | Lot C | Lot D |
|---|---|---|---|---|
| Core | | | | |
| Lercanidipine II | 30 | 30 | 30 | 30 |
| Surfactant | 1.3 | 1.3 | 1.3 | 1.3 |
| Osmotic agent | 100 | 100 | 100 | 100 |
| Binder | 22.7 | 22.7 | 22.7 | 22.7 |
| Filler | 50 | 43.2 | 35.2 | 20.2 |
| Water swellable polymer 1 | 68.4 | 68.2 | 68.2 | 68.2 |
| Water swellable polymer 2 | 5.7 | 5.7 | 5.7 | 5.7 |
| Glidant | 3.3 | 3.3 | 3.3 | 3.3 |
| Lubricant | 3.6 | 3.6 | 3.6 | 3.6 |
| Citric acid | 0 | 7 | 15 | 30 |
| Core weight | 285 | 285 | 285 | 285 |
| Membrane | | | | |
| Cellulose ester 1 | 28.45 | 28.45 | 28.45 | 28.45 |
| Cellulose ester 2 | 23.8 | 23.8 | 23.8 | 23.8 |
| Plasticizer | 2.75 | 2.75 | 2.75 | 2.75 |
| Membrane weight | 35 | 35 | 35 | 35 |

First, the core composition is prepared by placing lercanidipine hydrochloride, one water swellable polymer 1, a diluent, an osmotic agent, citric acid, the 50% of the glidant, and a binder in a high shear mixer and mix for 5 minutes. Then a water swellable polymer 2 is added and mixed for 1 minute more. The granulation process is initiated by the gradual addition of a granulating solution containing a surfactant and purified water to the high shear with continuous blending to produce a wet blend. Next, the wet blend is granulated and dried at 40-50° C. for 20 minutes in a static bed to remove the water. Then, the dry granules are screened through a 16 USP mesh screen for size reduction. Next, the screened granules are mixed with the 50% remaining of the glidant and a lubricant, that have been previously passed through a 40 mesh screen, in a V-Blender during 5 minutes. This final blend is tableted to provide the cores.

A first composition to cover the cores is prepared as follows: two cellulose esters and a plasticizer are added to organic solvent and purified water, and mixed thoroughly to form a polymer solution. This solution is sprayed onto the tablets in a perforated pan coater to form film-coated cores. A 0.5 mm hole is drilled through the coating to provide perforated film-coated tablets.

### EXAMPLE 2

Samples of lercanidipine/tartaric acid in a 1/1 ratio and lercanidipine/citric acid in a 1/1 ratio were prepared. One set of samples was stored in clear glass vials with butyl caps and aluminum seals and kept for 1.5 month at 50° C. A second set was put into vials and the butyl cap were replaced by gauze and exposed to 50° C.+75% RH while a third set of samples was maintained at 5° C. in refrigerator as reference condition. Analytical testing of the different samples was done by HPLC, column RP18, 300×3.9 mm ID, particle size 4 μm, in acetonitrile-0.15 M sodium perchlorate buffer, pH 3.0, at a flow rate of 1.3 ml/min, at 30° C., detector UV 240 nm. In all cases impurities were reported as % of the area of the impurity peak referred to the area of the main peak (a/a). The lercanidipine is the lercanidipine HCl polymorph II.

| | | | | Lercanidipine/ | | | Lercanidipine/ | | |
|---|---|---|---|---|---|---|---|---|---|
| | Lercanidipine | | | Tartaric Acid | | | Citric Acid | | |
| Type of Impurity | | | 50° C. + | | | 50° C. + | | | 50° C. + |
| (% a/a) | 5° C. | 50° C. | 75% RH | 5° C. | 50° C. | 75% RH | 5° C. | 50° C. | 75% RH |
| Impurity B | 0.02 | 0.01 | 0.01 | 0.01 | 0.01 | 0.09 | 0.01 | 0.01 | 0.22 |
| Impurity 3 | 0.18 | 0.22 | 0.28 | 0.05 | 0.09 | 4.85 | 0.06 | 0.10 | 0.50 |
| Maximum | nd | 0.03 | 0.05 | 0.01 | 0.03 | 0.77 | 0.12 | nd | 0.26 |
| unknown | | | | | | | | | |
| impurities | | | | | | | | | |
| Total impurities | 0.20 | 0.31 | 0.43 | 0.08 | 0.14 | 5.83 | 0.22 | 0.11 | 1.24 |

### EXAMPLE 3

An open label, randomized, cross-over, single-dose study of four periods is carried out according to Williams design with four formulations, three new lercanidipine 30 mg osmotic device formulations versus the marketed immediate release formulation Zanidip® 20 mg tablet (Recordati S.p.A., Italy). Twenty healthy male and female volunteers, from 45 to 65 years of age, will be given consecutive numbers from 1 to 20 according to the time they begin the study. Subjects will receive the 4 treatments during the study, with a washout period of at least 5 days in-between. The lercanidipine will be administered after an overnight fast, lasting al least 10 hours. Anamnesis, physical examination, vital signs, laboratory tests, ECG and adverse events recording will be performed. Treatment sequence will be assigned by randomization and thus each volunteer will receive any of the treatments under study in the first period, and the remaining in periods 2, 3 and 4 according to the allotted sequence. The evaluation of the plasma concentrations of lercanidipine will be at the following times after dose administration: 0 hours (preceding drug administration), 1, 1.5, 2, 2.5, 3, 4, 5, 6, 8, 10, 12, 14, 16, 20, 24 and 28 hours post-dose. The time of drug administration will be defined as study time Oh. The concentrations of lercanidipine in plasma will be determined by means of a validated LC-MS/MS method. The lower limit of quantification will be 0.1 ng/mL.

The preformed statistical analyses will be descriptive statistics on vital signs and PK parameters, ANOVA on log-transformed Cₘₐₓ, AUCₜ, AUC_{∞}, mean ratio (test/reference) and 90% confidence intervals for Cₘₐₓ, AUCₜ and AUC_{∞} and C₂₄ (not transformed values), and non-parametric 90% confidence intervals for tₘₐₓ median differences.

### EXAMPLE 4

Samples of lercardinidine/malic acid, lercarnidipine/ascorbic acid, lercanidipine/fumaric acid, lercanidipine/oxalic acid, and lercanidipine/succinic acid, each in a 1/1 ratio, were treated as described in example 2. The lercanidipine is the lercanidipine HCl polymorph II.

| Type of Impurity | | | 50° C. + | | | 50° C. + | | | 50° C. + |
|---|---|---|---|---|---|---|---|---|---|
| (% a/a) | 5° C. | 50° C. | 75% RH | 5° C. | 50° C. | 75% RH | 5° C. | 50° C. | 75% RH |
| | | | | Lercanidipine/ | | | Lercanidipine/ | | |
| | Lercanidipine | | | Malic Acid | | | Ascorbic Acid | | |
| Impurity B | 0.01 | 0.01 | 0.01 | nd | nd | 0.25 | 0.03 | 0.02 | 0.01 |
| Impurity 3 | 0.27 | 0.30 | 0.16 | 0.02 | 0.10 | 14.26 | 0.22 | 0.14 | 0.22 |
| Maximum | 0.04 | 0.04 | 0.04 | 0.04 | 0.04 | 1.22 | 0.04 | 0.04 | 0.05 |
| unknown | | | | | | | | | |
| impurities | | | | | | | | | |
| Total impurities | 0.45 | 0.47 | 0.34 | 0.15 | 0.24 | 23.37 | 0.44 | 0.33 | 0.43 |
| | Lercanidipine/ | | | Lercanidipine/ | | | Lercanidipine/ | | |
| | Fumaric Acid | | | Oxalic Acid | | | Succinic Acid | | |
| Impurity B | nd | 0.01 | 0.00 | nd | nd | 0.01 | 0.01 | 0.01 | 0.01 |
| Impurity 3 | 0.05 | 0.07 | 0.05 | 0.03 | 0.06 | 0.06 | 0.03 | 0.03 | 0.05 |
| Maximum | 0.04 | 0.06 | 0.04 | 0.04 | 0.04 | 0.04 | 0.04 | 0.04 | 0.04 |
| unknown | | | | | | | | | |
| impurity | | | | | | | | | |
| Total impurities | 0.19 | 0.27 | 0.18 | 0.17 | 0.29 | 0.21 | 0.17 | 0.19 | 0.21 |

### EXAMPLE 5

The following procedure is used to prepare osmotic device formulations containing lercanidipine (30 and 50 mg strength) in the core and lercanidipine HCl (10 mg strength) in an immediate or rapid release external drug-containing coat. The osmotic device formulations contain the following ingredients in the amounts indicated:

| Excipients/Functional category | Amount (mg) | |
|---|---|---|
| Lercanidipine ER strength | 30.00 | 50.00 |
| Lercanidipine IR/RR strength | 10.00 | 10.00 |

Core

| | | |
|---|---|---|
| Lercanidipine HCl (Polymorph II) | 30.00 | 50.00 |
| Surfactant | 1.00-30.00 | 2.00-50.00 |
| Diluent | 6.00-30.00 | 10.00-50.00 |
| Osmotic agent | 60.00-140.00 | 95.00-240.00 |
| Binder | 1.50-30.00 | 3.00-40.00 |
| Performance-enhancing acid | 0.90-40.00 | 2.00-65.00 |
| Osmopolymer 1 | 70.00-130.00 | 120.00-220.00 |
| Osmopolymer 2 | 0.00-40.00 | 0.00-70.00 |
| Osmopolymer 3 | 0.00-40.00 | 0.00-70.00 |
| Glidant | 0.10-10.00 | 0.20-15.00 |
| Lubricant | 0.50-15.00 | 1.00-25.00 |
| Purified water* | 20.00-50.00 | 30.00-80.00 |

Coating A

| | | |
|---|---|---|
| Cellulose ester 1 | 10.00-20.00 | 17.00-33.00 |
| Cellulose ester 2 | 0.00-17.00 | 0.00-28.00 |
| Plasticizer | 1.00-2.00 | 1.70-3.30 |
| Organic solvent* | 350.00-650.00 | 580.00-1080.00 |
| Purified water* | 60.00-130.00 | 100.00-220.00 |

Coating B (optional)

| | | |
|---|---|---|
| Water soluble polymer | 0.60-3.00 | 1.00-5.00 |
| Opaquant | 0.60-3.00 | 1.00-5.00 |
| Talc | 3.00-9.00 | 5.00-15.00 |
| Purified water* | 65.50-115.00 | 105.00-190.00 |

Coating C

| | | |
|---|---|---|
| Lercanidipine HCl (Polymorph II) | 10.00 | 10.00 |
| Film forming polymer | 10.00-40.00 | 10.00-40.00 |
| Plasticizer | 1.50-6.00 | 1.50-6.00 |
| Disintegrant | 4.00-15.00 | 4.00-15.00 |
| Glidant | 0.25-1.00 | 0.25-1.00 |
| Purified water* | 250.00-1000.00 | 250.00-1000.00 |

Coating D

| | | |
|---|---|---|
| Film forming polymer | 7.00-14.00 | 10.00-20.00 |
| Plasticizer | 0.07-3.50 | 0.10-5.00 |
| Opaquant | 0.70-3.50 | 1.00-5.00 |
| Talc | 0.70-7.00 | 1.00-10.00 |
| Purified water* | 126.00-210.00 | 180.00-300.00 |

*denotes a component used during manufacture of the osmotic device but which is substantially absent (present in an amount of less than about 10% or less than 5% by wt.) in the final dosage form.

Diluent, osmopolymers 1 and 2, a half of the glidant and the binder are first individually screened using a Quadro Comil equipped with a 0.075 inch screen. The osmotic agent and the performance-enhancing acid are milled using a Fitz Mill equipped with a screen 0.033 inch. The previous ingredients and the lercanidipine are placed in a high shear granulator bowl and mixed during 5 minutes to obtain an homogenous powder blend. The osmopolymer 3 is individually screened using a Quadro Comil equipped with a 0.075 inch screen, and then is added to the homogenous powder blend and mixed during 1 additional minute. The granulation process is initiated by the gradual addition of the surfactant in purified water to form a solution that is then added to the homogenous powder blend in order to obtain a consistent granulation end point. The wet granules are sieved through a Quadro Comil equipped with a 0.375 inch screen and then dried in a static bed oven for reducing the humidity content between 1.0-2.5%. Next, the dry granules are milled using a Quadro Comil equipped with a 0.125 inch screen and then with a 0.045 screen in order to reduced and homogenize particle size. Next, a mixture of the other half of the glidant and the lubricant, previously sieved through a 0.017 inch screen, is added and mixed for about 5 minutes to obtain the final blend in a V blender. This final blend is compressed in a Rotary Tablet Press with 9.25-11.00 mm diameter punches to obtain uncoated cores. The average weight of the uncoated cores is approximately between 276 to 294 mg for the 30 mg dose and between 460 to 490 mg for the 50 mg dose.

An osmotic coating (A) composition is prepared as follows: organic solvent and purified water are charge into a suitable vessel. Under continuous stirring, cellulose esters 1 and 2 are added and stirred vigorously until dissolution is completed. The plasticizer is added and homogenized. The mixture is sprayed onto the uncoated cores to obtain coated cores. The membrane coating of each core is then perforated with laser equipment to form at least one passageway of 0.2-0.8 mm through the semipermeable coat.

The second coating (B) is prepared by mixing the water soluble polymer, opaquant and talc in purified water. This polymer mixture is sprayed onto the tablets in a perforated pan coater to obtain film-coated tablets which membrane coating weighs approximately 13 mg for the 30 mg dose and 21 mg for the 50 mg.

The third coating (C) is prepared by mixing the lercanidipine, the plasticizer, the film forming polymer, the glidant and the disintegrant in purified water to obtain an homogeneous suspension. This previous mixture is sprayed onto the tablets in a perforated pan coater or fluid bed dryer in order to obtain film-coated tablets which membrane coating weighs 46 mg approximately for both strength.

A final coating composition (D) is prepared as follows: the plasticizer, the film forming polymer, the opaquant and the talc are mixed in water. This mixture is sprayed onto the tablets in a perforated pan coater to obtain film-coated tablets which membrane coating weighs approximately 15 mg for the 30 mg dose and 24 mg for the 50 mg.

### EXAMPLE 6

The following procedure was used to prepare osmotic device formulations containing lercanidipine and an acidifying agent. The osmotic device formulations contained the following ingredients in the amounts indicated:

| | |
|---|---|
| Ingredient | Amount (mg) |

Core

| | |
|---|---|
| Lercanidipine hydrochloride (Polymorph I) | 30.00 |
| Polisorbate 20 (Tween 20) | 1.30 |
| Microcrystalline cellulose PH 101 | 33.70 |
| Sodium chloride | 101.50 |
| PVP K 30 | 22.70 |
| Polyethylene oxide WSR 205 | 68.20 |
| Performance-enhancing acid** | 15.00 |
| Hydroxypropylmethylcellulose (2208) K 4 M | 5.70 |
| Coloidal silicon dioxide | 3.30 |
| Magnesium stearate | 3.60 |
| Purified water* | 285.00 |

Coating

| | |
|---|---|
| Cellulose acetate 320 S NF | 31.00 |
| Cellulose acetate 398 10 NF | 26.00 |
| Polyethylene glycol 400 | 3.00 |
| Acetone* | 1020.00 |
| Purified water* | 180.00 |

*denotes a component used during manufacture of the osmotic device but which is substantially absent (present in an amount of less than about 10% or less than 5% by wt.) in the final dosage form.
**The following performance-enhancing acids were used to prepare five different formulations: oxalic acid, succinic acid, fumaric acid, citric acid and tartaric acid.

First, the core composition was prepared by placing lercanidipine hydrochloride, polyethylene oxide WSR 205, microcrystalline cellulose PH 101, sodium chloride, a performance-enhancing acid, the 50% of colloidal silicon dioxide, and PVP K 30 in a high shear mixer and mix for 5 minutes. Then hydroxypropylmethylcellulose was added and mixed for 1 minute more. The granulation process was initiated by the gradual addition of a granulating solution containing Tween 20 and purified water to the high shear with continuous blending to produce a wet blend. Next, the wet blend was granulated and dried at 40-50° C. for 20 minutes in a static bed to remove the water. Then, the dry granules were screened through a 16 USP mesh screen for size reduction. Next, the screened granules were mixed with the 50% remaining of the colloidal silicon dioxide and magnesium stearate, that had been previously passed through a 40 mesh screen, in a V-Blender during 5 minutes. This final blend was tableted to provide the cores.

A first composition to cover the cores was prepared as follows: cellulose acetate 320 S NF, cellulose acetate 398 10 NF and polyethylene glycol 400 were added to acetone and purified water, and mixed thoroughly to form a polymer solution. This solution was sprayed onto the tablets in a perforated pan coater to form film-coated cores. A 0.5 mm hole was drilled through the coating to provide perforated film-coated tablets.

### EXAMPLE 7

An open label, randomized, cross-over, multi-dose study under fed condition, three periods with two new formulations, lercanidipine osmotic device formulations of example 8 versus the marketed immediate release formulation Zanidip® 20 mg tablet (Recordati S.p.A., Italy) is carried out. Healthy male and female volunteers, from 21 to 65 years of age, are given consecutive numbers according to the time they begin the study. Subjects receive the 3 treatments during the study, with a washout period of at least 5 days in-between. Each new formulation is administered once daily, with standardized breakfast after an overnight fast along seven days. Anamnesis, physical examination, vital signs, laboratory tests, ECG and adverse events recording is performed. Treatment sequence is assigned by randomization and thus each volunteer receives any of the treatments under study in the first period, and the remaining in periods 2 and 3 according to the allotted sequence.

The evaluation of the plasma concentrations of lercanidipine are evaluated at sequential times after dose administration on the last day of treatment administration. The concentrations of lercanidipine in plasma is determined by means of a validated LC-MS/MS method. The lower limit of quantification is 0.1 ng/mL.

The preformed statistical analyses are descriptive statistics on vital signs and PK parameters, ANOVA on log-transformed Css (Cmin and Cmax), AUCt, AUCinf, mean ratio (test/reference) and 90% confidence intervals for Css (Cmin and Cmax), AUCt and AUCinf (not transformed values).

### EXAMPLE 8

The following procedure is used to prepare osmotic device formulations containing lercanidipine HCl (30 and 50 mg strength based upon lercanidipine) in the core and lercanidipine HCl (10 mg strength based upon lercanidipine) in an immediate or rapid release external drug-containing coat. The osmotic device formulations contain the following ingredients in the amounts indicated:

| | |
|---|---|
| Ingredients | Amount (mg) |

| | | |
|---|---|---|
| Lercanidipine ER strength | 30.00 | 50.00 |
| Lercanidipine IR/RR strength | 10.00 | 10.00 |

Core

| | | |
|---|---|---|
| Lercanidipine HCl (Polymorph II) | 30.00 | 50.00 |
| Polysorbate 20 | 1.30 | 2.17 |
| Microcrystalline cellulose 101 | 8.70 | 14.50 |
| Sodium chloride | 100.00 | 166.70 |
| Polyvinylpyrrolidone K 30 | 22.70 | 37.83 |
| Fumaric acid | 7.00 | 11.70 |
| Polyethylene oxide WSR 205 | 99.80 | 166.30 |
| Hydroxypropylmethylcellulose 100 LV | 4.30 | 7.17 |
| Hydroxypropylmethylcellulose K4M | 4.30 | 7.17 |
| Colloidal silicon dioxide | 3.30 | 5.50 |
| Magnesium stearate | 3.60 | 6.00 |
| Purified water* | 34.20 | 56.40 |

Coating A

| | | |
|---|---|---|
| Cellulose acetate 320 S | 15.38 | 25.00 |
| Cellulose acetate 398 10 NF | 8.50 | 14.00 |
| Polyethylene glycol 400 | 1.50 | 2.50 |
| Acetone* | 510.00 | 830.00 |
| Purified water* | 90.00 | 160.00 |

Coating B (optional)

| | | |
|---|---|---|
| Copolyvidone | 3.00 | 5.00 |
| Titanium Dioxide | 2.90 | 4.80 |
| Talc | 6.30 | 10.40 |
| Purified water* | 65.50 | 106.80 |

Coating C

| | | |
|---|---|---|
| Lercanidipine HCl (Polymorph II) | 10.00 | 10.00 |
| Hydroxypropylmethylcellulose 2910 | 24.00 | 24.00 |
| Polyethylene glycol 6000 | 3.50 | 3.50 |
| Crospovidone | 8.00 | 8.00 |
| Colloidal silicon dioxide | 0.50 | 0.50 |
| Purified water* | 540.00 | 540.00 |

Coating D

| | | |
|---|---|---|
| Copolyvidone | 12.75 | 20.00 |
| Polyethylene glycol 400 | 0.75 | 1.20 |
| Titanium dioxide | 0.75 | 1.20 |
| Talc | 0.75 | 1.20 |
| Purified water* | 126.00 | 200.00 |

*denotes a component used during manufacture of the osmotic device but which is substantially absent (present in an amount of less than about 10% or less than 5% by wt.) in the final dosage form.

Microcrystalline cellulose 101, hydroxypropylmethylcellulose K4M and hydroxypropylmethylcellulose 100 LV, a half of the colloidal silicon dioxide and the polyvinylpyrrolidone K 30 are first individually screened using a Quadro Comil equipped with a 0.075 inch screen. Sodium chloride and fumaric acid are milled using a Fitz Mill equipped with a screen 0.033 inch. The previous ingredients and lercanidipine are placed in a high shear granulator bowl and mixed during 5 minutes to obtain an homogenous powder blend. The polyethylene oxide WSR 205 is individually screened using a Quadro Comil equipped with a 0.075 inch screen, and then is added to the homogenous powder blend and mixed during 1 additional minute. The granulation process is initiated by the gradual addition of the Polysorbate 20 in purified water to form a solution that is then added to the homogenous powder blend in order to obtain a consistent granulation end point. The wet granules are sieved through a Quadro Comil equipped with a 0.375 inch screen and then dried in a static bed oven for reducing the humidity content between 1.0-2.5%. Next, the dry granules are milled using a Quadro Comil equipped with a 0.125 inch screen and then with a 0.045 screen in order to reduced and homogenize particle size. Next, a mixture of the other half of the colloidal silicon dioxide and the magnesium stearate, previously sieved through a 0.017 inch screen, is added and mixed for about 5 minutes to obtain the final blend in a V blender. This final blend is compressed in a Rotary Tablet Press with 9.25-11.00 mm diameter punches to obtain uncoated cores. The average weight of the uncoated cores is approximately 280 mg for the 30 mg dose and approximately 475 mg for the 50 mg dose.

An osmotic coating (A) composition is prepared as follows: acetone and purified water are charge into a suitable vessel. Under continuous stirring, cellulose acetate 320 S and cellulose acetate 398 10 NF are added and stirred vigorously until dissolution is completed. Polyethylene glycol 400 is added and homogenized. The mixture is sprayed onto the uncoated cores to obtain coated cores. The membrane coating of each core is then perforated with laser equipment to form at least one passageway of 0.2-0.8 mm through the semipermeable coat.

The second coating (B) is prepared by mixing copolyvidone, titanium dioxide and talc in purified water. This polymer mixture is sprayed onto the tablets in a perforated pan coater to obtain film-coated tablets which membrane coating weighs approximately 13 mg for the 30 mg dose and 21 mg for the 50 mg.

The third coating (C) is prepared by mixing lercanidipine, polyethylene glycol 6000, hydroxypropylmethylcellulose 2910, colloidal silicon dioxide and crospovidone in purified water to obtain an homogeneous suspension. This previous mixture is sprayed onto the tablets in a perforated pan coater in order to obtain film-coated tablets which membrane coating weighs approximately 46 mg for both strength.

A final coating composition (D) is prepared as follows: polyethylene glycol 400, copolyvidone, titanium dioxide and the talc are mixed in water. This mixture is sprayed onto the tablets in a perforated pan coater to obtain film-coated tablets which membrane coating weighs approximately 15 mg for the 30 mg dose and approximately 24 mg for the 50 mg dose.

The above is a detailed description of particular embodiments of the invention. It is recognized that departures from the disclosed embodiments may be made within the scope of the invention and that obvious modifications will occur to a person skilled in the art. Those of skill in the art should, in light of the present disclosure, appreciate that many changes can be made in the specific embodiments which are disclosed herein and still obtain a like or similar result without departing from the spirit and scope of the invention. All of the embodiments disclosed and claimed herein can be made and executed without undue experimentation in light of the present disclosure.

1. An osmotic device comprising: 1) a core comprising lercanidipine, or a pharmaceutically acceptable salt thereof, a performance-enhancing acid, and one or more other pharmaceutical excipients; and 2) a wall enveloping the core and comprising at least one preformed passageway, wherein the device provides a controlled release of lercanidipine over a period of 8-36 hours.

2. The osmotic device of claim 1, wherein the core thereof comprises lercanidipine, or a pharmaceutically acceptable salt thereof, a performance-enhancing acid, an osmotic agent, and at least one swelling polymer, and optionally one or more pharmaceutical excipients.

3. The device of claim 1, wherein the osmotic device provides an increased bioavailability of lercanidipine following administration of the osmotic device to a subject in need thereof as compared to a control osmotic device excluding the performance-enhancing acid.

4. The device of claim 1, wherein the osmotic device provides an increased stability of lercanidipine in the osmotic device during storage as compared to a control osmotic device excluding the performance-enhancing acid.

5. The device of claim 1, wherein the osmotic device provides an increased rate of release of lercanidipine upon exposure of the device to an aqueous environment of use or following administration to a subject in need thereof as compared to an otherwise similar (control) osmotic device excluding the performance-enhancing acid.

6. The device of claim 1, wherein the osmotic device provides an increase in the overall amount of lercanidipine released upon exposure of the device to an aqueous environment of use or following administration to a subject in need thereof as compared to an otherwise similar control osmotic device excluding the performance-enhancing acid.

7. The device of claim 1, wherein lercanidipine is present as a mineral acid salt and the performance-enhancing acid is present as an organic acid.

8. The device of claim 7, wherein the performance-enhancing acid comprises a monocarboxylic acid, dicarboxylic acid, tricarboxylic acid, hydroxy-carboxylic acid, hydroxy-dicarboxylic acid, hydroxy-tricarboxylic acid, nonaromatic organic acid, or a combination thereof.

9. The device of claim 7, wherein the mineral acid is hydrochloric acid and the performance-enhancing acid is a dicarboxylic acid.

10. The device of claim 7, wherein the performance-enhancing acid is selected from the group comprising fumaric acid, oxalic acid, and succinic acid.

11. The device of claim 1 comprising 30-60 mg of lercanidipine.

12. The device of claim 1 further comprising an immediate release coating comprising lercanidipine external to the wall.

13. The device of claim 12, wherein the immediate release coating comprises 10 mg of lercanidipine.

14. The device of claim 1, wherein the wall is a semipermeable membrane.

15. The device of claim 1 further comprising at least one other pharmaceutically active agent.

16. The device of claim 15, wherein the at least one other pharmaceutically active agent is selected from the group consisting of an angiotensin converting enzyme inhibitor, an angiotensin II receptor blocker, a β-blocker, an α-blocker, a diuretic, and a combination thereof.

17. The device of claim 16, wherein the angiotensin converting enzyme inhibitor is selected from the group consisting of enalapril, captopril, lisinopril, benazepril, enalaprilat, espirapril, fosinopril, moexipril, quinapril, ramipril, perindopril, and trandolapril.

18. The device of claim 16, wherein the angiotensin II receptor blocker is selected from the group consisting of olmesartan, irbesartan, valsartan, telmisartan, losartan and eprosartan.

19. The device of claim 16, wherein the β-blocker is selected from the group consisting of carvedilol, pindolol, propranolol, practolol, metoprolol, esmolol, oxprenolol, timolol, atenolol, alprenolol, sotalol, carteolol, nadolol, betaxolol, penbutolol, acebutolol, and bisoprolol.

20. The device of claim 16, wherein the α-blocker is selected from the group consisting of doxazosin, prazosin, terazosin, and labetalol.

21. The device of claim 16, wherein the diuretic is selected from the group consisting of chlorothiazide, acetazolamide, methazolamide, triamterene, furosemide, indapamide, flumethiazide, bumetanide, ethacrynic acid, torsemide, muzolimide, azosemide, piretanide, tripamide, hydrochlorothiazide, chlorthalidone, metozalone, cyclopenthiazide, amiloride, xipamide, mefruside, dorzolamide, ethoxzolamide, cyclothiazide, clopamide, dichlorphenamide, hydroflumethiazide, trichlormethiazide, polythiazide and benzothiazide.

22. The device of claim 15, wherein the at least one other pharmaceutically active agent is selected from the group consisting of: enalapril maleate; enalapril maleate and hydrochlorothiazide; lisinopril; lisinopril and hydrochlorothiazide; olmersartan; olmersartan and hydrochlorothiazide; irbesartan; irbesartan and hydrochlorothiazide; carvedilol; carvedilol and hydrochlorothiazide; doxazosin; and doxazosin and hydrochlorothiazide.

23. The device according to claim 1, wherein the lercanidipine is released from the core according to the following approximate release profile:

| | |
|---|---|
| Time | Range (%) |
| (hrs) | Min | Max |
|---|---|---|
| 0 | 0 | 0 |
| 0.5 | 15 | 21 |
| 1 | 18 | 26 |
| 3 | 20 | 31 |
| 9 | 43 | 66 |
| 15 | 62 | 86 |
| 24 | 78 | 100 |

24. The device according to claim 1, wherein the performance-enhancing acid is present in the range of about 2.5%-15% wt. based upon the weight of the uncoated core.

25. The device according to claim 1 further comprising adsorbent, antioxidant, buffering agent, colorant, flavorant, sweetening agent, antiadherent, binder, diluent, direct compression excipient, disintegrant, glidant, lubricant, opaquant, polishing agent or a combination thereof.

26. The device according to claim 1, wherein the wall comprises plural preformed passageways.

27. The device according to claim 1, wherein the wall comprises plural grades of cellulose acetate.

28. A method of treating a disease or disorder that is therapeutically responsive to lercanidipine comprising administering to a subject in need thereof an osmotic device according to claim 1.

29. (canceled)

30. (canceled)

31. (canceled)

32. (canceled)

33. (canceled)

34. (canceled)

35. (canceled)
